(19) United States
(12) Patent Application Publication
(10) Pub. No.: US 2026/0216060 A1
(43) Pub. Date: Jul. 30, 2026

(54) COMPOSITIONS FOR GLUCOSE-RESPONSIVE DRUG DELIVERY SYSTEMS VIA MULTIFUNCTIONAL HYDROGEL-BASED MICRONEEDLE

(71) Applicant: THE GOVERNING COUNCIL OF THE UNIVERSITY OF TORONTO, TORONTO (CA)

(72) Inventors: XIAOYU WU, TORONTO (CA); AMIN GHAVAMI NEJAD, TORONTO (CA); FULE LIU, RICHMOND HILL (CA); BRIAN LU, TORONTO (CA); ADRIA GIACCA, TORONTO (CA); SAKO MIRZAIE, TORONTO (CA); MELISA SAMARIKHALAJ, TORONTO (CA)

(21) Appl. No.: 19/120,328

(22) PCT Filed: Oct. 16, 2023
(86) PCT No.: PCT/CA2023/051373

§ 371 Date: Apr. 11, 2025

Related U.S. Application Data
(60) Provisional application No. 63/416,141, filed on Oct. 14, 2022.
(60) Provisional application No. 63/416,657, filed on Oct. 17, 2022.

(51) Int. Cl. A61K 9/00; A61K 38/26; A61K 38/28; A61P 3/10

(52) U.S. Cl. CPC A61K 9/0021; A61K 38/26; A61K 38/28; A61P 3/10

(57) ABSTRACT

The present disclosure provides a matrix microneedle patch made of a hyaluronic acid (HA) polymeric backbone functionalized with needle height dopamine (DA) and 4-amino-3-fluorophenylboronic acid (AFBA, pKa~7.5) that quickly and spontaneously crosslinks upon mixing of the polymer solutions by auto-oxidation of catechol groups and reversible interactions between AFBA and catechol functional groups in the absence of any chemical crosslinking agent. The DA and AFBA content were selected for conjugation into the backbone of the HA polymer for the desired hormone delivery profile. The patch provides high drug loading capacity for long-term drug delivery application. The crosslinking mechanism for microneedle fabrication is biocompatible and beneficial for sustaining hormone drug stability and bioactivity as it does not require harsh crosslinking conditions. Facile pH adjustment of the matrix hydrogel can be easily casted into a microneedle patch without multistep processes that conventional patch polymerization requires. The present matrix microneedle patch demonstrates sufficient skin penetration, rapid swelling in interstitial media, high drug loading capacity and effective hypo/hyperglycemia prevention by the automated hypo/hyperglycemia-triggered delivery of hormones through the skin.

### FIELD

The present disclosure relates to compositions of multifunctional hydrogel-based microneedle patches that are prepared by a novel self-crosslinking strategy for delivering blood glucose-reducing therapeutic agent in response to high blood glucose levels and blood glucose-raising therapeutic agents in response to low blood glucose levels.

### BACKGROUND

It is estimated that about 463 million people worldwide are living with diabetes in 2019, which is projected to rise to 578 million in 2030. About 10% of people with type 1 diabetes (T1D) rely on insulin therapy for survival, and ~30% of those with type 2 diabetes (T2D) use insulin for glycemic control and preventing diabetic complications. However, intensive insulin therapy associated with hypoglycemia causes a serious problem and life-threatening condition in T1D; weight gain, insulin resistance and various side effects diminish the benefit of insulin therapy in T2D. Current therapies provide support but require constant monitoring of blood glucose levels and administration of insulin or counteracting/correcting hormones by patients or caregivers, which are tedious or even impossible for rescuing patients under severe hypoglycemia who are alone, too young, too old, confused, or unconscious.

Hypoglycemia or the state of abnormally low blood glucose levels (≤70 mg/dL) is the most serious acute complication associated with insulin therapy that can result in death if left untreated. Hypoglycemia is currently treated by rescue/emergency glucagon kits or nasal powder spray. However, such emergency/rescue treatments still require someone to assist the patient and thus, are not ideal for patients that suffer from hypoglycemia unawareness or those under severe hypoglycemia who are alone, juvenile, elderly, confused, or unconscious. Hence, there is a clear need for preventative therapies of hypoglycemia. To date, two strategies have been employed to reduce the risk of hypoglycemia, including the use of glucose-responsive insulin analogs and systems that deliver insulin, glucagon and its derivatives glucose-responsively. Glucose-responsive insulin analogs have not yet passed clinical trials due in part to the possibility of non-specific binding (other than the receptor) of the formulated insulin analogs. Many glucose-responsive insulin and glucagon delivery systems have also been attempted via microneedle patches (MNs), and electromechanical closed-loop systems as well, but without much clinical success.

Improved glycemic control currently relies on hybrid closed-loop insulin delivery systems. The system combines a continuous glucose monitoring device connected to an insulin pump that delivers insulin based on the blood glucose level. However, patients need to wear such systems 24 hours a day with tethered pumps and tubing. Wearing such devices poses many risks, such as skin infections from the cannula and diabetic ketoacidosis due to probable device malfunctions.

Nonetheless, the expensive devices still require patients' intervention managing their blood sugar and changing the cannula. This cumbersome maintenance if often further complicated by unpredictable diet and exercise patterns. Most importantly, inaccurate administration of excess insulin can inevitably cause hypoglycemia. Thus, there is an important need for an automated glucose-responsive system that integrates both sensing and delivery components that self-regulate to automatically release blood glucose reducing agent (e.g., insulin) in response to high blood glucose levels.

### SUMMARY

The present disclosure provides smart matrix microneedle patches, made of a hyaluronic acid polymeric backbone functionalized with dopamine (DA) and 4-amino-3-fluorophenylboronic acid (AFBA) that quickly and spontaneously crosslinks upon mixing of the polymer solutions by auto-oxidation of catechol groups and reversible interactions between AFBA and catechol functional groups in the absence of any chemical crosslinking agent. This dual crosslinking strategy has been used to synthesize many different smart hydrogels; however, it has never been used to fabricate any kind of glucose-responsive MN patches. The DA and AFBA content was rationally tailored for conjugation onto the backbone of the HA polymer and the ratio of DA-HA to AFBA-HA was optimized for the desired glucose-responsive swelling and hormone delivery profiles.

The MN patch can provide high drug loading capacity and capability of stabilizing the protein/peptides which are important for long-term drug delivery application. The introduced crosslinking mechanism for the microneedle fabrication is more biocompatible (less toxic) than the conventional crosslinking methods that use elevated temperatures or ultraviolet irradiation; and therefore, beneficial for sustaining hormone drug stability and bioactivity. A facile pH adjustment of the matrix hydrogel can be easily introduced during the cast of a microneedle array without the hassle of multistep processes required by conventional polymerization method. This novel matrix microneedle patch demonstrates sufficient skin penetration, rapid swelling in interstitial media, high drug loading capacity and effective hyperglycemia prevention by the automated hyperglycemia-triggered delivery of hormones through the skin.

Thus, the present disclosure provides a method of producing a matrix microneedle patch, comprising mixing polymer solutions of natural and saccharide-based polymeric backbone functionalized with catechol containing material and glucose-sensing component which quickly and spontaneously crosslinked by auto-oxidation of catechol groups and reversible interactions between the glucose-sensing component and catechol functional groups in the absence of any chemical crosslinking agent.

The matrix microneedle patch may comprise a glucose-sensing component, wherein polymers containing the glucose-responsive moieties shows higher interaction with glucose in response to high glucose level causing swelling of the microneedle and rapid release of the blood glucose-reducing agent.

The matrix patch comprise of glucose-sensing component may dissociate with glucose in response to lower glucose levels and favorably interact with catechol components, which lead to the release of a blood glucose-raising agent such as Zinc-Glucagon (Z-GCN)) in response to hypoglycemia.

The saccharide-based polymeric component may be a Hyaluronic acid (HA), its derivatives and other saccharide-based polymers such as alginate.

The microneedle component is suitable to stabilize the native structure of the therapeutic agent.

The microneedles are crosslinked upon mixing of the polymer solutions at a specific ratio.

The glucose-responsive moiety (“glucose-sensing component”) may be a boronic acid-containing compound such as phenylboronic acid and its derivatives.

The therapeutic agent is a peptide hormone that increases and/or reduces the blood glucose.

The peptide hormone is glucagon or insulin analogues.

The blood glucose-reducing therapeutic agent may be a peptide hormone that reduces the blood sugar.

The peptide hormone may be Insulin or Insulin analogues.

The glucose-reducing agent may be GLP-1, or GLP-1 analogues.

The glucose-reducing agent may be pramlintide.

The glucose-reducing agent may be gama-aminobutyric acid.

The glucose-reducing agent may be glucose-dependent insulinotropic polypeptide or GLP.

The glucose-raising agent is metal containing therapeutics such as Zinc-Glucagon.

The present disclosure provides a matrix microneedle patch device for treating high blood sugar or preventing low blood sugar, comprising a base; an array of microneedles extending from, and away, from said base, each microneedle of said array and said base being made of a crosslinked mixture of natural and/or synthetic polymeric backbones, catechol containing molecules conjugated to a first preselected fraction of the natural and/or synthetic polymeric backbones and glucose-sensing molecules containing glucose-responsive moieties conjugated to a second preselected fraction of the natural and/or synthetic polymeric backbones with the first and second fractions being crosslinked via catechol-catechol crosslinkages and catechol-glucose-sensing molecules crosslinkages in an absence of chemical crosslinking agents; and blood glucose-reducing agents entrapped within the crosslinked polymeric backbones or blood glucose-raising agents non covalently bonded to catechol moieties in the catechol containing molecules.

The present disclosure provides a matrix microneedle patch device having contained therein a composition for treating high blood sugar or preventing low blood sugar, compromising:
a microneedle patch with a base;
an array of microneedles extending from and away from said base;
blood glucose-raising agents or blood glucose-reducing agents loaded into the patch;
each microneedle of said array and said base being made of a mixture of natural and/or synthetic polymeric backbones;
catechol containing molecules covalently conjugated to the natural and/or synthetic polymeric backbones, wherein, the catechol is either covalently or non-covalently coupled to one another that form crosslinkages between the polymeric backbones;
glucose-sensing molecules containing glucose-responsive moieties covalently conjugated to the natural and/or synthetic polymeric backbones that interact with catechol containing molecules, wherein the glucose-responsive moieties reversibly interact or dissociate with the catechol containing molecules response of the concentration in the of a diol-containing compound; and
wherein when blood glucose-reducing agents are present they are entrapped and prevents blood glucose-raising beyond a preselected blood glucose level, and when the blood glucose-reducing agents are present they are non-covalently bonded to catechol moieties in the catechol containing molecules.

In an embodiment there is provided a method of producing a matrix microneedle patch containing blood glucose-reducing agents or blood glucose-raising agents, comprising:
providing a mold of preselected size for receiving polymer solutions therein, which once the patch is produced it has a base section and microneedles extending away from said base section;
mixing a first polymer solution of natural and/or synthetic polymeric backbones having catechol containing molecules covalently conjugated to the polymeric backbones with blood glucose-reducing agents or blood glucose-raising agents and a second polymer solution of natural and/or synthetic polymeric backbones containing glucose-responsive moieties covalently conjugated to the polymeric backbones that interact with the catechol containing molecules such that the natural and/or synthetic polymeric backbones crosslink by auto-oxidation of catechol groups and reversible interactions between the glucose-sensing molecule and the catechol functional groups in the absence of any chemical crosslinking agent; and
wherein when blood glucose-reducing agents are present they are entrapped and prevent the blood glucose level rising beyond a preselected blood glucose level, and when the blood glucose-increasing agents are present they are non-covalently bonded to catechol moieties in the catechol containing molecules and increase blood glucose levels and prevents reduction of blood glucose beyond a preselected blood glucose level. The natural and/or synthetic polymeric backbones are crosslinked upon mixing of the polymer solutions at a specific weight ratio, wherein a weight ratio of the catechol-conjugated natural and/or synthetic polymers to glucose-sensing molecules conjugated natural and/or synthetic polymeric backbones are about 0.25:3 to about 3:0.25. Alternatively, the weight ratio is about 1.1 to about 3:1. Alternatively, weight ratio is about 2:1.

A further understanding of the functional and advantageous aspects of the disclosure can be realized by reference to the following detailed description and drawings.

### BRIEF DESCRIPTION OF THE DRAWINGS

### Section A

Embodiments will now be described, by way of example only, with reference to the drawings, in which:

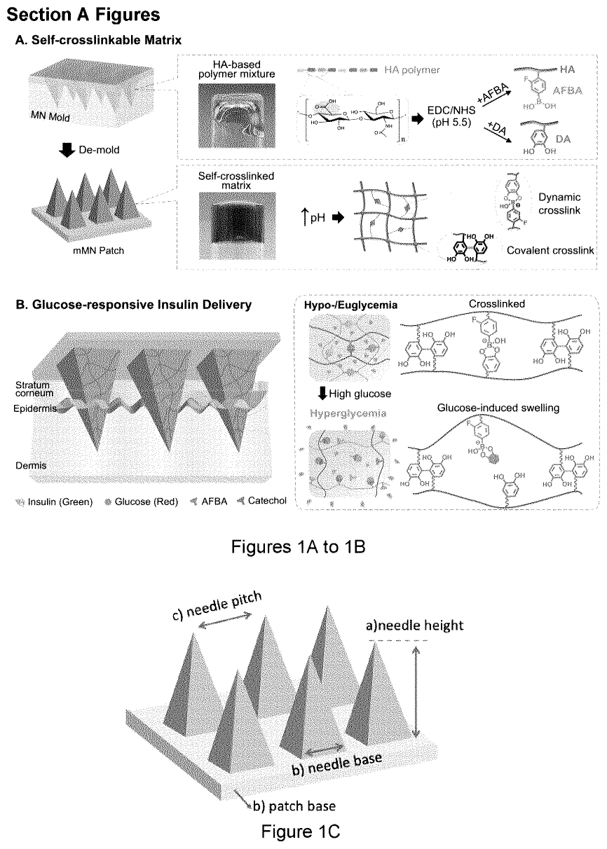

FIG. 1A shows a schematic diagram of matrix patch fabrication, polymer synthesis, and crosslinking mechanism. The polymer mixture for mMN fabrication is comprised of HA polymer functionalized with AFBA and DA via EDC/NHS coupling reaction. The self-crosslinked mMN is formed by raising the pH to a slightly alkaline condition. The matrix contains both dynamic and covalent crosslinks within the hydrogel network. Dynamic crosslinking (reversible) is the association between the catechol group of DA and AFBA, whereas covalent crosslinking (irreversible) is attributed to the DA-DA linkage.

FIG. 1B shows a schematic diagram of matrix patch application through the skin and the mechanism of insulin release from the patch at hyperglycemia. Under hypo/eu-glycemic conditions, the boronate of AFBA forms a complex with catechol, and insulin is physically trapped within the matrix network. In the presence of high glucose, AFBA preferably forms a complex with glucose by dissociating with catechol groups. This uncomplexation breaks the dynamic crosslinked network, which enhances the hydrogel swelling and promotes insulin release.

FIG. 1C is a side view of the matrix microneedle patch device as disclosed herein showing the base and the microneedles extending away from the base, with the microneedles having a length or height a, a width b of the base of the needle, a pitch or distance between adjacent needles being c, a width d of the base of the patch.

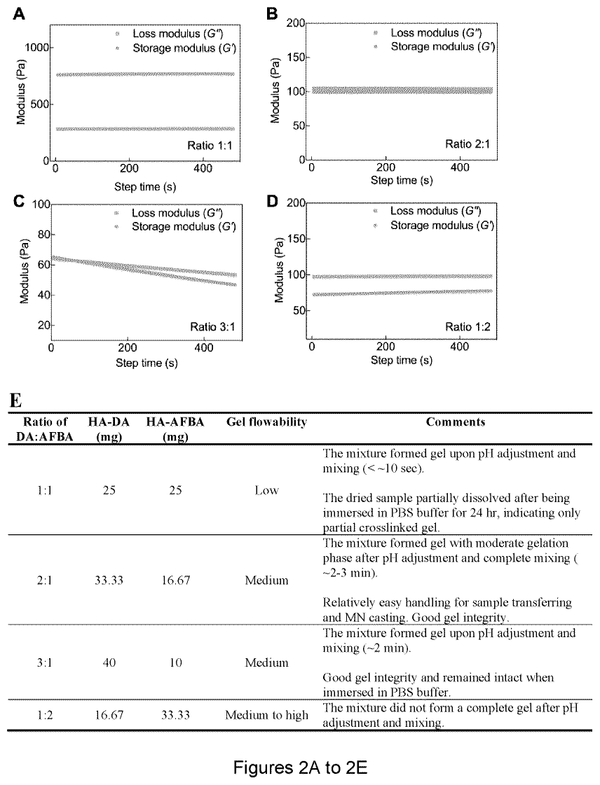

FIG. 2A shows rheometric analysis of matrix hydrogel formed with weight ratios of 1:1 HA-DA to HA-AFBA, in a time sweep mode after gelation induction.

FIG. 2B shows rheometric analysis of matrix hydrogel formed with weight ratios of 2:1 HA-DA to HA-AFBA, in a time sweep mode after gelation induction.

FIG. 2C shows rheometric analysis of matrix hydrogel formed with weight ratios of 3:1 HA-DA to HA-AFBA, in a time sweep mode after gelation induction.

FIG. 2D shows rheometric analysis of matrix hydrogel formed with weight ratios of 1:2 HA-DA to HA-AFBA, in a time sweep mode after gelation induction.

FIG. 2E shows the evaluation of gel flowability using various ratios (w/w) of HA-DA and HA-AFBA polymers in different weight ratios.

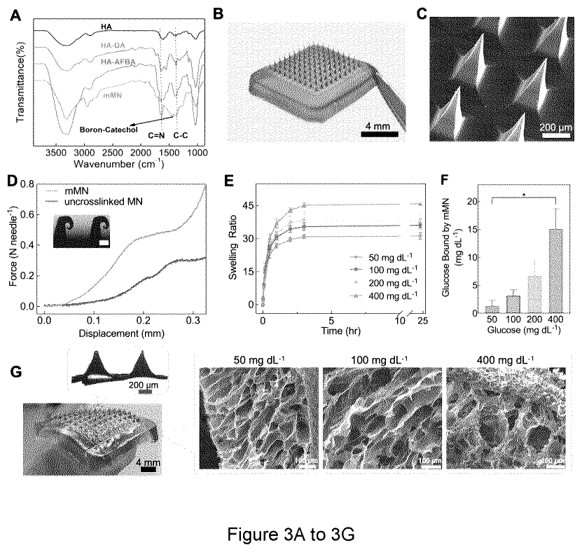

FIG. 3A shows the FTIR spectra of HA, HA-DA, HA-AFBA and matrix microneedle array (mMN) made from a mixture of HA-DA and HA-AFBA.

FIG. 3B shows a photograph of the mMN patch with sharp needles.

FIG. 3C shows a typical SEM image of the mMN tips with sharp needles.

FIG. 3D shows stress-strain curves of the mMN patch under compression before and after crosslinking.

FIG. 3E shows swelling ratio of the mMN in pH 7.4 PBS at various glucose concentrations (50, 100, 200, or 400 mg dL-1), representing hypoglycemic, euglycemic, and hyperglycemic conditions.

FIG. 3F shows Glucose-binding capability of the mMN in PBS at various glucose concentrations (50, 100, 200, or 400 mg dL-1), representing hypoglycemic, euglycemic, and hyperglycemic conditions.

FIG. 3G shows Photograph of swollen mMN (left) and SEM images of the cross-section of mMN after swollen at various glucose concentrations (50, 100, 200, or 400 mg dL-1) (right).

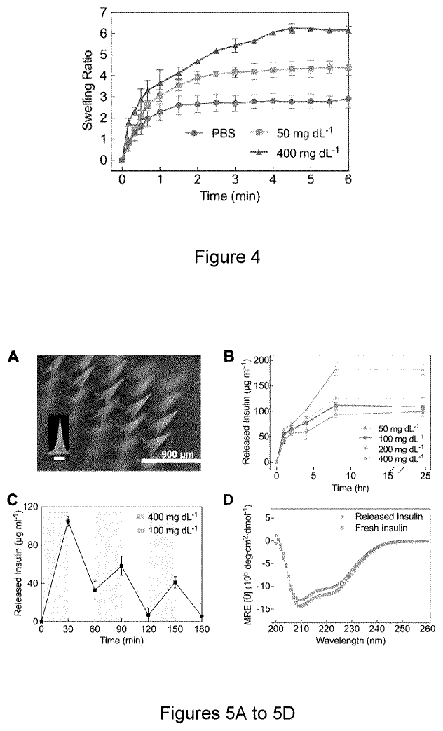

FIG. 4. Glucose-dependent swelling profile of the mMN tips determined by the volume change of MNs prior to and at the predetermined time points after inserting the needles into a 1.4 wt % agarose hydrogel. The data presented as mean±standard deviation (n=3 and n=2 for data point of 400 mg dL⁻¹ after 2 min due to the microneedles swelled beyond the microscope lens).

FIG. 5A shows Fluorescence microscopy image of mMN array loaded with FITC-Insulin, (inset: a magnified image of the microneedle, scale bar: 200 μm).

FIG. 5B shows In vitro accumulated glucose-responsive release of insulin from mMNs in PBS at various glucose concentrations (50, 100, 200, or 400 mg dL-1), representing hypoglycemic, euglycemic, and hyperglycemic conditions.

FIG. 5C Shows Pulsatile release profile of insulin from mMN patches at various glucose concentrations (50, 100, 200, or 400 mg dL-1), representing hypoglycemic, euglycemic, and hyperglycemic conditions.

FIG. 5D shows Circular dichroism spectra of insulin released from the mMN compared to freshly prepared insulin solution.

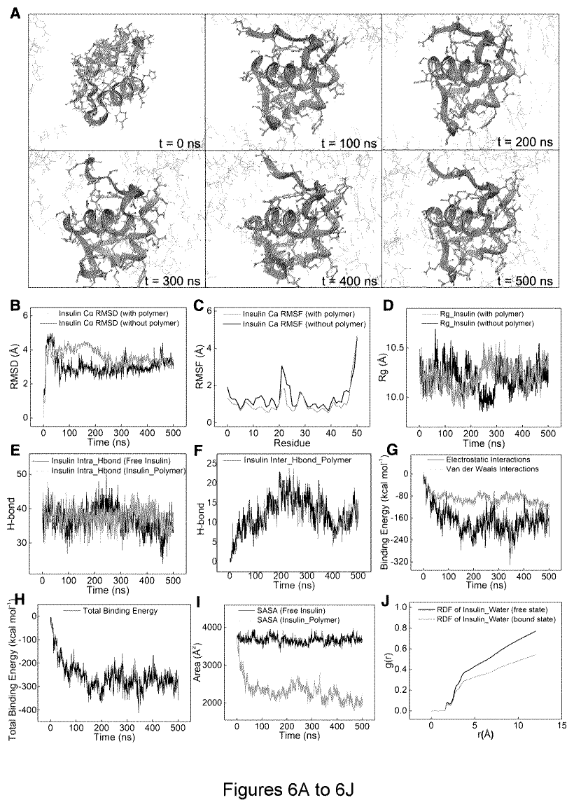

FIG. 6A shows the snapshots of the insulin/polymer system at various times, t, during the 500 ns MD simulation. Insulin secondary structure is depicted in blue/green ribbons.

FIG. 6B shows the Insulin root-mean-square deviation in the absence and presence of the polymer.

FIG. 6C shows the Insulin root-mean-square fluctuation in the absence and presence of the polymer.

FIG. 6D shows the Insulin radius of gyration in the absence and presence of the polymer.

FIG. 6E shows the results extracted from MD simulations of insulin in the absence and presence of the polymer showing the H-bonding between the polymer and insulin.

FIG. 6F the results extracted from MD simulations of insulin in the absence and presence of the polymer showing Intermolecular H-bonds between insulin and the polymer.

FIG. 6G shows the results extracted from MD simulations of insulin in the absence and presence of the polymer showing the electrostatic, Van der Waals and total binding energy between insulin and the polymer.

FIG. 6H shows the results extracted from MD simulations of insulin in the absence and presence of the polymer showing the total binding energy between insulin and the polymer.

FIG. 6I shows the results extracted from MD simulations of the solvent accessible surface area of insulin in the absence and presence of the polymer.

FIG. 6J shows the results extracted from MD simulations about radial distribution function of water molecules around free insulin and polymer-bound insulin.

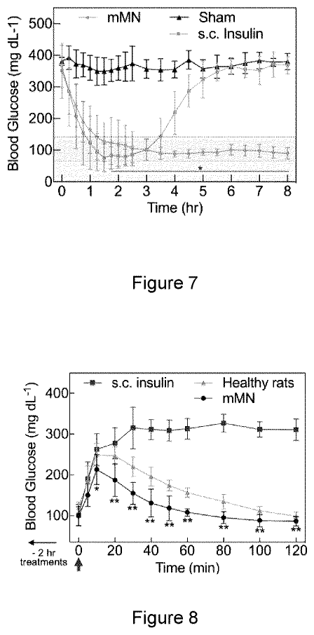

FIG. 7 shows the In vivo insulin efficacy of mMN patches versus sham patch and s.c. injected insulin solution.

FIG. 8 shows the In vivo intraperitoneal glucose tolerance test (IPGTT) in diabetic rats.

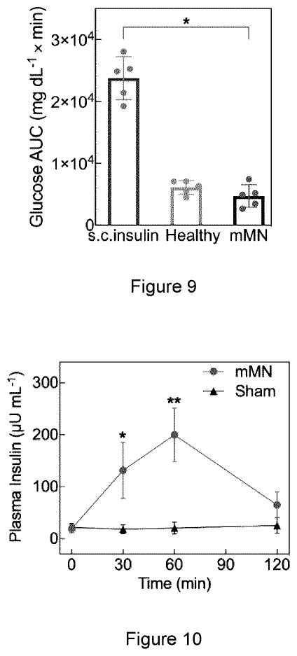

FIG. 9 shows the area under the curve (AUC) of glucose responses in T1D rats during IPGTT with the baseline set to blood glucose level at 0 min.

FIG. 10 shows the plasma insulin levels at t=0 min, 60 min, and 120 min of the IPGTT.

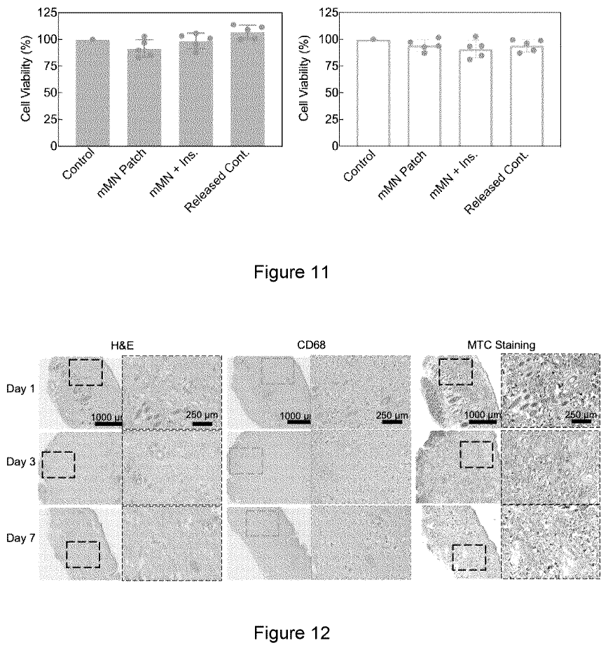

FIG. 11 shows in vitro cytotoxicity study of the matrix microneedle patch components on (A) NIH-3T3 fibroblast cells and (B) HaCaT Human keratinocyte cells. mMN: sham patch, mMN+Ins.: sham patch+insulin, released pellet: components released from the insulin-loaded mMN. All data is shown as mean±standard deviation, n=5.

FIG. 12 shows the H&E-stained (left) and CD68-stained (middle) and Masson's trichrome (MTC, right) staining results. The treated rat skin tissue was harvested on day 1, day 3, and day 7 post-mMN patch removals. Inset images with black/red box sections are enlarged views of the box sections on the left.

### Section B Figures

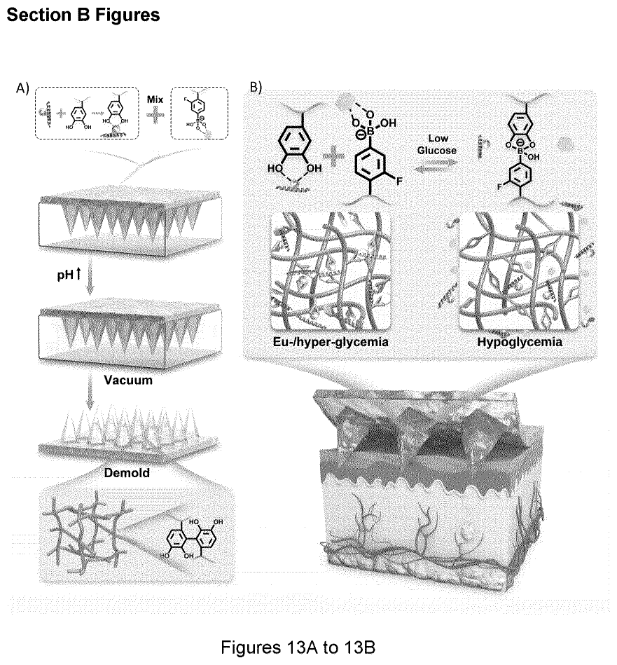

FIG. 13A shows a schematic diagram of matrix patch fabrication;

FIG. 13B shows mechanism of zinc-glucagon release;

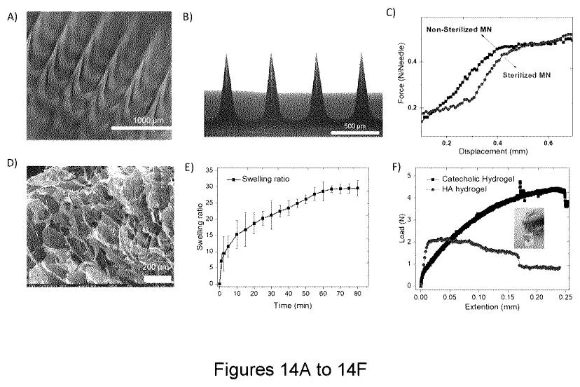

FIG. 14A shows a 3D image of transmittance microscopic images of microneedle array;

FIG. 14B shows a side view of the transmittance microscopic images of microneedle array of FIG. 2A;

FIG. 14C shows mechanical compression strength of the MN patch before and after sterilization by gamma irradiation;

FIG. 14D shows SEM images elucidating the inner morphology/porosity of fabricated patch;

FIG. 14E shows swelling ratio of the MN patch after immersing in phosphate buffer solution;

FIG. 14F shows the force-displacement curves of adhesion of the synthesized hydrogel to the glass slides coated with a medical tape to mimic skin like texture; inset shows a photograph of the hydrogel adhered on skin's finger. the data presented as mean±standard deviation (n=3);

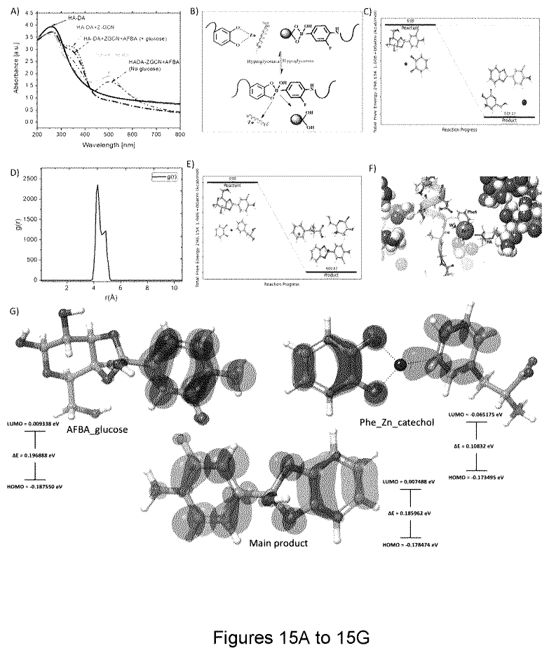

FIG. 15A UV-Vis measurements of HA-DA, HA-DA+ZGCN, HADA+AFBA, and HADA+ZGCN+AFBA in the presence and absence of glucose;

FIG. 15B shows a schematic illustration of the Z-GCN catechol and AFBA interactions in presence of high/low glucose concentrations.

FIG. 15C shows reaction energy profile of reactants (zinc-catechol and glucose-AFBA complexes) and products (zinc ion, glucose, and AFBA-catechol). (D) Radial distribution function (RDF) as a function of distance between zinc ion and Phe 6 from GCN. (E) Reaction energy profile of reactants (Phe-zinc-catechol and glucose-AFBA complexes) and products (Phe-zinc, glucose, and AFBA-catechol);

FIG. 15D shows the radial distribution function (RDF) as a function of distance between zinc ion and Phe 6 from GCN;

FIG. 15E shows the reaction energy profile of reactants (Phe-zinc-catechol and glucose-AFBA complexes) and products (Phe-zinc, glucose, and AFBA-catechol);

FIG. 15F shows the depiction of the interaction mode among zinc Ion, solvent molecules, polymer, and GCN. In this illustration, the water molecules are denoted by ‘W.’ Interacting residues are represented using a stick model, while the non-interacting portions of the polymer and glucagon are shown through a ball model and cartoon presentation, respectively;

FIG. 15G shows the density surface plots for the highest occupied molecular orbital (HOMO) and lowest unoccupied molecular orbital (LUMO) of glucose-AFBA, Phe-zinc-catechol, and catechol-bound AFBA;

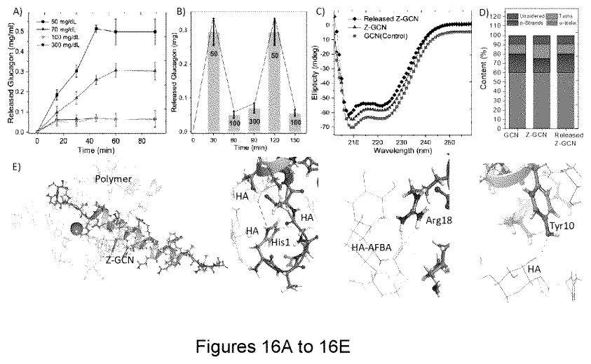

FIG. 16A shows the in vitro glucose-responsive release of Z-GCN from the MN patch in PBS mimicking hypoglycemia (50 mg/dL), euglycemia (100 mg/dL) or hyperglycemia (300 mg/dL) (n=3);

FIG. 16B shows the pulsatile release of Z-GCN from the MN patch at various glucose concentrations (50, 100, 200, or 400 mg dL-1), representing hypoglycemic, euglycemic, and hyperglycemic conditions;

FIG. 16C shows the CD spectra of native glucagon solution and released Z-GCN;

FIG. 16D shows the deconvolution of CD spectra to reveal fractions of glucagon secondary structure;

FIG. 16E shows the calculated interaction mode between Z-GCN and the polymer, as determined by clustering analysis. Z-GCN and polymer residues are shown as ball-and-stick model and wire representation;

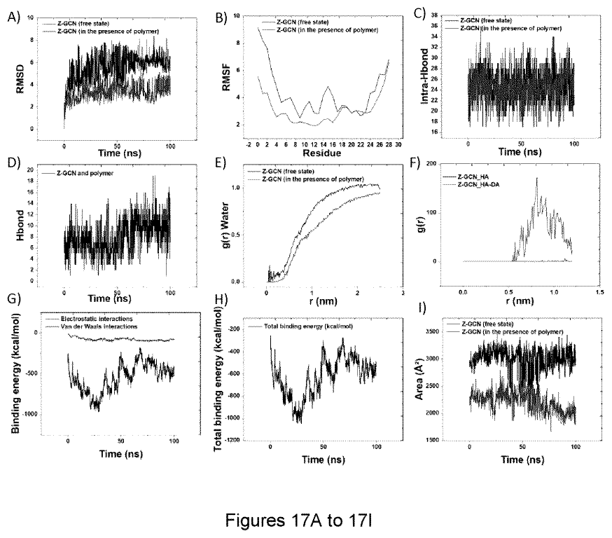

FIG. 17A shows the Z-GCN root-mean-square deviation;

FIG. 17B shows the Z-GCN root-mean-square fluctuation;

FIG. 17C shows the Z-GCN intra-molecular H-bonding;

FIG. 17D shows the H-bonding between Z-GCN and polymer. (E) RDF between water molecules and Z-GCN;

FIG. 17F shows the RDF between Z-GCN and polymer residues;

FIG. 17G shows the electrostatic interaction and Van der Waals binding energies between Z-GCN and polymer;

FIG. 17H shows the total binding energies between Z-GCN and polymer;

FIG. 17I shows the solvent-accessible surface area (SASA) of Z-GCN in the absence and presence of the polymer;

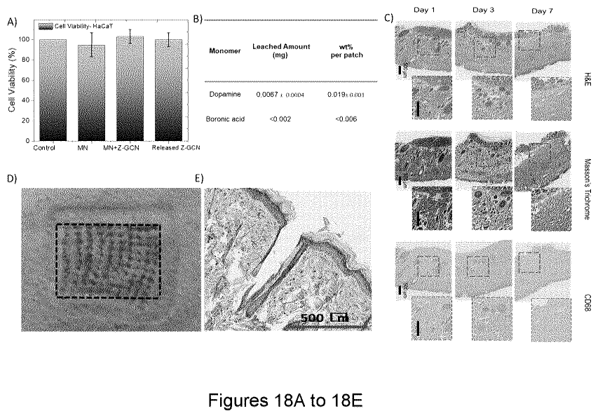

FIG. 18A shows the (A) In vitro cytotoxicity study of the MN, MN containing Z-GCN and released Z-GCN in HaCaT keratinocytes.

FIG. 18B shows the estimated amount of leachable comonomers released from the MN patch as detected by RP-HPLC;

FIG. 18C shows histological staining of rat skin up to 7 days post-patch application. The MN patch was applied on shaven rat skin for 12 hours and removed thereafter. The treated area of the skin was excised one (day 1), three (day 3), and seven (day 7) days post-patch removal. Excised skin was stained with H&E, MTC, and CD68 antibody. Dashed red line boxes show an area with higher magnification, scale bars=500 μm. No significant signs of skin inflammation were observed across seven days post-patch application;

FIG. 18D shows photograph of MN insertion through the stratum corneum;

FIG. 18E shows histological analysis of H&E-stained tissue section of rat skin following Trypan blue dye loaded-MN insertion through the stratum corneum. F

FIG. 18A shows the average blood glucose level after administration of 1 mg GCN vs 1 mg of Z-GCN; The average blood glucose response of rats treated either by sham patch or Z-GCN (1 mg) containing patch after an insulin challenge;

FIG. 18B shows in-vivo study design of hypoglycemia prevention study;

FIG. 18C shows the average blood glucose level after patch application (t=0 min). Data is shown as mean±standard deviation, n=3, an asterisk indicates a p-value <0.05;

FIG. 18D shows plasma glucagon levels as determined by glucagon ELISA. Data are represented as mean±standard deviation, n=5, ** indicates a p-value <0.01 and * indicates a p-value <0.05 to signify statistically significant difference between groups.

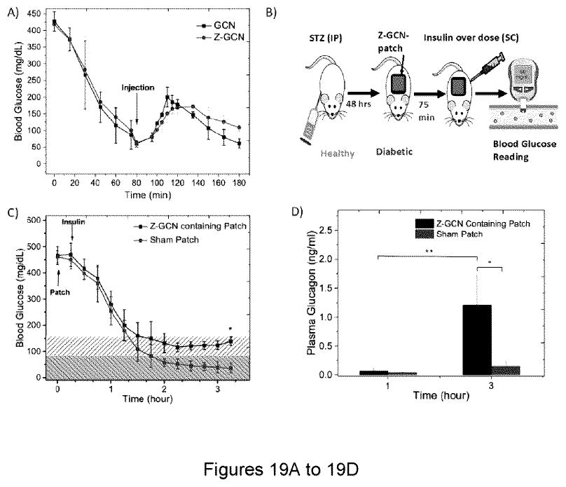

FIG. 19A shows in vivo efficacy and hypoglycemia prevention study against an insulin overdose. The average blood glucose level after administration of 1 mg GCN vs 1 mg of Z-GCN; The average blood glucose response of rats treated either by sham patch or Z-GCN (1 mg) containing patch after two insulin challenges;

FIG. 19B shows in-vivo study design of hypoglycemia prevention study,

FIG. 19C shows the average blood glucose level after patch application (t=0 min). Data is shown as mean±standard deviation, n=3, an asterisk indicates a p-value <0.05;

FIG. 19D shows plasma glucagon levels as determined by glucagon ELISA. Data is represented as mean±standard deviation, n=5, * indicates a p-value <0.01 and ** indicates a p-value <0.05 to signify statistically significant difference between groups.

### DETAILED DESCRIPTION

Various embodiments and aspects of the disclosure will be described with reference to details discussed below. The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

As used herein, the terms “comprises” and “comprising” are to be construed as being inclusive and open ended, and not exclusive. Specifically, when used in the specification and claims, the terms “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the terms “about” and “approximately” are meant to cover variations that may exist in the upper and lower limits of the ranges of values, such as variations in properties, parameters, and dimensions. Unless otherwise specified, the terms “about” and “approximately” mean plus or minus 25 percent or less.

It is to be understood that unless otherwise specified, any specified range or group is as a shorthand way of referring to each and every member of a range or group individually, as well as each and every possible sub-range or sub-group encompassed therein and similarly with respect to any sub-ranges or sub-groups therein. Unless otherwise specified, the present disclosure relates to and explicitly incorporates each and every specific member and combination of sub-ranges or sub-groups.

As used herein, the term “on the order of”, when used in conjunction with a quantity or parameter, refers to a range spanning approximately one tenth to ten times the stated quantity or parameter.

As used herein the “operably connected”, “operably associated” or “operably attached” means that the two elements are connected or attached either directly or indirectly. Accordingly, the items need not be directly connected or attached but may have other items connected or attached therebetween.

This disclosure describes a novel glucose-responsive hydrogel-based microneedle system that is designed for the autonomous and minimally-invasive glucose-responsive delivery of hormones to achieve tight glycemic control (within the normoglycemic window) in people with diabetes. The advantage of mussel-inspired (catechol-based) chemistry were used to design a novel hydrogel-based MN patch capable of encapsulating highly concentrated hormone drug, super swelling, and rapid delivery of insulin at hyperglycemia.

FIG. 1C, as noted above, shows a side view of the matrix microneedle patch device as disclosed herein showing the base and the microneedles extending away from the base, with the microneedles having a length or height a, a width b of the base of the needle, a pitch or distance between adjacent needles being c, and a width d of the base of the patch.

The matrix microneedle patch device is comprised of a backing layer, a base on the backing layer and an array of needles projecting away from the based. All parts of matrix microneedle patch device are made from the same materials. In a preferred embodiment the matrix microneedle patch device is caste in a mold as a single unitary one-piece item. The patch device may be made in different sizes depending on the size of the mold used to make the device. The base may have a thickness in a range from about 50 to about 2000 micrometers, the needle height may be in a range from about 200 to about 1000 micrometers, the needle pitch may be in a range from about 500 to about 1500 micrometers, where the needle pitch is distance between the centers of any two needles on the same needle base. The microneedle patch may have an array size: of anywhere from 1×1 to 100×100 needles on the base, where the array size is the number of needles in each patch.

The microneedles may be made be cast using a mold as mentioned above, it will be appreciated that other methods may be used to produce the matrix microneedle patch device, such as but not limited to 3D printing, micromachining, lithography and droplet-air blowing and electro-drawing.

### Examples

The following non-limiting examples are presented to enable those skilled in the art to understand and to practice embodiments of the present disclosure. They should not be considered as a limitation on the scope of the disclosure, but merely as being illustrative and representative thereof.

The specific embodiments described above have been shown by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

### Novel Hydrogel-Based Microneedle Patch

The smart matrix microneedle patch is made of a hyaluronic acid polymeric backbone functionalized with dopamine (DA) and 4-amino-3-fluorophenylboronic acid (AFBA) that can be quickly crosslinked upon mixing of aqueous solutions of the polymers at slightly alkaline pH. The crosslinking of mMN is formed via (i) covalent catechol-catechol (DA-DA) linkages and (ii) the dynamic/reversible complexation between catechol and AFBA without the need of an additional crosslinking agent and/or application of heat or UV light. This dual crosslinking strategy has been used to synthesize many different smart hydrogels; however, it has never been used to fabricate any kind of smart MN patches. The DA and AFBA content were rationally selected for conjugation into the backbone of the HA polymer for the desired hormone delivery profile. The patch can provide high drug loading capacity that is important for long-term drug delivery application.

The introduced crosslinking mechanism for the microneedle fabrication is more biocompatible (less toxic) and beneficial for sustaining hormone drug stability and bioactivity as it does not require harsh crosslinking conditions, i.e., exposure to elevated temperatures or ultraviolet irradiation, that is conventionally used for fabricating hydrogel-based microneedles. A facile pH adjustment of the matrix hydrogel can be easily casted into a microneedle patch without the hassle of multistep processes that conventional patch polymerization requires. This novel matrix microneedle patch demonstrated sufficient skin penetration, rapid swelling in interstitial media, high drug loading capacity and effective hypo/hyperglycemia prevention by the automated hypo/hyperglycemia-triggered delivery of hormones through the skin.

### Section A

### A Novel Glucose-Responsive Mechanism of Insulin Delivery in a Microneedle System

The complexation of catechol-AFBA not only plays a role in the crosslinking process but also provides a glucose-responsive property to the mMN patch. The latter stems from the glucose level-dependent complexation between catechol and boronate groups. Under physiological and hyperglycemic conditions, AFBA preferably binds to glucose by dissociating with the catechol groups, leading to the breaking of the secondary crosslinked network, enhancing the swelling of the hydrogel and promoting insulin release (FIGS. 1A and 1B). Conversely, under hypo/normoglycemic conditions, AFBA reversibly forms a complex with catechol, reforming its secondary crosslinked network to promote hydrogel densification, thereby retarding insulin release and reducing the risk of hypoglycemia. This novel mMN patch demonstrated sufficient skin penetration, rapid and ultra-high swelling capability, high insulin loading capacity, and effective hypo/hyper-glycemia prevention. Owing to its excellent insulin delivery profile at hyperglycemia and capability of preserving the bioactivity of insulin, the proposed MN patch is of great potential for minimally-invasive administration of insulin, as confirmed via in vivo experiments.

This is the first demonstration of a self-crosslinkable and glucose-responsive matrix microneedle system fabricated from a simple and mild crosslinking method to regulate blood glucose levels automatically by delivering hormones glucose-responsively. Although microneedle patches have been published and patented for diabetes management (Web of Science: 85), the amount of knowledge for using self-crosslinkable, glucose-responsive, and matrix-based microneedle patches for diabetic management cannot be found in literature. Only a handful of matrix-based microneedle patches have been reported for glucose control in diabetes (Web of Science: 10). However, most prior published works used thermal or ultra-violet irradiation containing organic solvents, chemical crosslinkers, and thermal/photoinitiators to fabricate matrix MN patches. Moreover, the conjugation of AFBA as the glucose-responsive component in matrix microneedles has not yet been reported.

Furthermore, regarding simple methods for fabricating microneedle systems, only two technologies have been reported with the first is disclosed in Gu et al (2019) (US Provisional Patent Application No. 617/270,953) have developed a method to fabricate glucose-responsive matrix microneedle patch in situ for insulin delivery. However, the system was formed via in situ polymerization and crosslinked in organic solvent using a conventional method that requires ultraviolet irradiation for 30 minutes. Such a method created excessive unreacted residues (e.g., monomers, crosslinkers and photoinitiators) left behind, causing toxic effects when entering the body after patch administration. Even though such a system was immersed in phosphate buffered saline containing 20% ethanol solution for purification, the system still had a tracible amount of unreacted monomers that were detected. Unwanted drug release (~10%) also occurred during the purification step. Moreover, the bioactivity of loaded protein may be reduced by such polymerization conditions.

The second is disclosed in Chen et al (2021) recently reported a mild preparation method was developed to fabricate glucose-responsive microneedle patch for insulin delivery. Such a system utilized the borate ester bond with polyvinyl alcohol to form a pre-hydrogel solution without polymerization and organic solvents. However, the absence of covalent crosslink of such system did not truly achieve glucose-dependent drug release in vitro and lack in vivo evidence for its pulsatile release behavior. Moreover, the pre-hydrogel also needs to be treated with a freeze/thaw process 3 times to make crystalline crosslinking of polyvinyl alcohol chains, which is inconvenient.

As previously mentioned, at the heart of the introduced system is a rationally-designed self-crosslinkable and glucose-responsive hydrogel and successfully formulating this functional polymer required various supporting innovations. Some of the key points are listed below:

Conjugated PBA monomers onto HA polymer with organic solvent had a low conjugation efficiency due to its poor solubility in water. Choosing a suitable water-based solvent was of crucial importance for this system, and a water-ethanol mixture at a specific ratio was found to improve boronate conjugation efficiency.

The ratio between DA and AFBA conjugates in the composition of the HA-based hydrogel was rationally selected for the optimal glucose-responsive release profile while forming a stable non-degradable 3D matrix network to provide clinically relevant drug loading capacity for effective prolonged glycemic control.

The numbers of AFBA conjugates can drastically change the hydrogel characteristics. For instance, it was found that low AFBA conjugation ratio had unstable gelation outcomes, weakening the integrity of matrix MN patches. Too high or low degree of DA conjugation also affects self-crosslinking efficiency and its ability for MN fabrication.

A composition for preventing hypoglycemia: comprising a hydrogel-based MN that self-crosslinked via catechol-metal interaction.

A composition for delivering blood glucose reducing agents at hypoglycemia condition: wherein the crosslinking method does not influence the bioactivity of blood glucose-reducing therapeutic agent.

A composition for delivering insulin at hyperglycemia condition: comprising a hydrogel containing the containing boronic acid groups that competitively bind with and Boronic acid and catechol causing rapid release of the blood glucose-reducing therapeutic agent.

Synthesis of Multifunctional HA Polymer with Dopamine (DA) and AFBA

HA-DA and HA-AFBA were synthesized by carbodiimide coupling reaction. The chemical structure of HA-DA and HA-AFBA were characterized using ¹H-NMR. The N-acetyl peak of HA appeared at 1.9 ppm. Multiplets from 3 to 3.8 ppm were associated with disaccharide units in the HA backbone. Chemical shift at δ=6.7 to 7.32 ppm corresponds to the catechol aromatic ring of dopamine. The multiplets from δ=7.29 to 7.8 ppm corresponded to the benzene ring of AFBA. The degree of catechol conjugation in the HA backbone is important in controlling the density of polymer crosslink.

The amount of dopamine in HA conjugates was confirmed using UV-absorbance at 280 nm wavelength with dopamine standards. The absorbance of dopamine increases as the HA-DA conjugates increases while not observed in HA alone. HA-DA conjugates with various dopamine feed ratios were tested for the desired gelation time. The degree of substitution of DA was calculated using the 1H-NMR spectrum. The DA conjugation (HA-DA1) at 16% was further investigated for microneedle fabrication, given its relatively moderate gelation action. The AFBA in HA-AFBA conjugates was also verified using the UV-Vis absorbance at a wavelength of 250 nm with AFBA standards. The feeding ratio of AFBA to HA backbone is 0.65 mM of AFBA per 1 g of HA. The degree of AFBA conjugation was confirmed to be around 12.9% using the ¹H-NMR spectrum. UV-Vis spectrum of both HA-DA and HA-AFBA at its corresponded absorbance confirmed the conjugation of dopamine and AFBA to HA via amide bonds was accomplished.

### Optimization of Polymer Ratio Prior to Patch Fabrication

It is important to mix the polymers in a proper ration to fabricate a hydrogel with appropriate rheological property for patch fabrication. Therefore, the rheology of HA-DA and HA-AFBA mixtures at various weight ratios was studied to identify a desired gelation kinetic that is suitable for the patch fabrication while achieving glucose-responsiveness (FIGS. 2A to 2E). Among the tested ratios, HA-DA and HA-AFBA at a 2:1 ratio was selected for fabrication of the mMN patch due to the relatively slower gelation phase, easier transferring of the hydrogel to the mold, and better integrity of formed MNs with sharp tips.

Fabrication and Characterization of the Self-Crosslinked MN Matrix Patch (mMN) for Glucose-Responsive Delivery of Blood Glucose-Reducing Therapeutic Agent

The mMN patch was fabricated based on the catechol oxidation method and performed at room temperature. In brief, polymer mixture was prepared by dissolving various ratios (w/w) of HA-DA and HA-AFBA in DDI water. Lyophilized insulin was solubilized in 0.1 M NaOH solution and thoroughly mixed into the polymer mixture. The amount of solvent used in insulin solution was factored into the polymer and solvent ratio. The pH of the polymer mixture was raised from 6.5 to 7.8. Upon drastic color change, the mixed gel was cast into a polydimethylsiloxane (PDMS) microneedle (MN) mold. Afterward, the mold was placed in a vacuum under 25 mmHg for 5 min to remove trapped air. The crosslinked mixture was left air dry at room temperature, and the dried mMN patch was separated from the mold and stored in a desiccator until use, see FIGS. 3B & 3C. The uncrosslinked MN patch was prepared similarly but without the pH adjustment.

### Chemical Characterization

### FTIR Method

The bonding configuration of the polymer samples and mMN patch were identified by attenuated total reflectance (ATR)-Fourier transform infrared (FTIR) spectroscopy. The IR spectra were recorded at room temperature using a Paragon 1000 spectrometer (Perkin Elmer) equipped with VeeMax II variable angle ATR accessory.

### Result

Fourier transform infrared (FTIR) spectroscopy was used to confirm the successful crosslinking of mMN patch, see FIG. 3A. Compared to HA, HA-DA and HA-AFBA show two peaks at 1591, and 1285 cm⁻¹, attributed to C═N stretching vibrations and aromatic C—C stretching vibrations, respectively, indicating that DA and AFBA functional groups were successfully conjugated onto the HA backbone. The IR spectrum of polymers after crosslinking shows a new peak at 1480 cm⁻¹ due to the formation of the catechol-boronate complex. Furthermore, this sample shows a significant reduction in the broadband at around 3300-3400 cm⁻¹ which corresponds to the covalent DA-DA linkage.

### Mechanical Strength

The mechanical strength of mMN patches was determined by compression test using Instron 3366 universal testing machine with a compression load cell. The MN array (tips facing upwards) was placed flat on a compression plate. A vertical force was applied perpendicularly at a constant speed of 0.5 mm min⁻¹ to the MN patch using a flat-head stainless steel probe. The displacement was measured until the MN tips began to buckle. The initial distance between the base of MN patch and the flat head of the probe was set at 2 mm, with a cell loading capacity set at 10 N. Instantaneous load (force; N) and displacement (distance; mm) were recorded by the testing machine every 0.05 s to generate the load-displacement curve. The force-at-break was recorded as the needle began to buckle.

### Results

The force-at-break of the crosslinked mMN patch was over 0.45 N needle⁻¹ compared with 0.27 N needle⁻¹ for the uncrosslinked MN patch, see FIG. 3D, demonstrating that crosslinking provided an adequate mechanical strength for skin penetration in the following animal studies.

### Swelling Studies

### Method

The swelling capability of mMN patch was determined by immersing mMN in pH 7.4 PBS containing varying glucose concentrations (50, 100, 200 or 400 mg dL⁻¹) at 34° C. The net weight of the swelled mMN patch was carefully measured at predetermined time points. The swelling ratio was calculated based on a formula of the weight of the mMN patches at various time (Wₜ) to its initial net weight (W₀) as shown below.

$$Swelling Ratio = \frac{{W}_{t} - {W}_{0}}{{W}_{0}}$$

The swelling kinetics of mMN tips were evaluated by inserting the MNs into a translucent agarose gel (1.4 wt %). The subsequent volume change of the mMN tips with respect to the time of exposure to agarose gel was recorded by a microscope equipped with a CCD camera. The volume change of mMN tips was determined using ImageJ software. The swelling ratio was calculated based on a formula of the volume of the mMN tips at a different time (Vt) to its initial volume (Vo) as shown below. The internal structure of swollen hydrogels was cryo-dried and lyophilized before being examined by SEM.

$$Swelling Ratio = \frac{{V}_{t} - {V}_{0}}{{V}_{0}}$$

### Result:

As shown in FIG. 3E, the mMN patch swelled rapidly once immersed in the PBS solution and showed a glucose concentration-dependent swelling behavior. The mMN became extremely swollen at hyperglycemia and reached over 45 times of its dry weight after 3 hr of immersing in the glucose media at 400 mg dL-1.1 It should be noted that the needles of the swellable mMN remained intact at the maximum swelling state (FIG. 3G). Moreover, a similar swelling trend was obtained by observing the changes in the volume of mMN tips after insertion into a glucose-containing agarose gel (FIG. 4). Such a high swelling ratio is consistent with previous observations for dynamically associative crosslinked hydrogels. The mechanism of glucose-dependent swelling of the mMN is further supported by a glucose binding efficiency study. The amount of glucose bound to mMN through the AFBA groups in hyperglycemic media (400 mg dL⁻¹) was 4.8-fold greater than that in the euglycemic media (100 mg dL⁻¹) (SEM images revealed, see FIG. 3G, the internal microstructures/porosity of the swollen mMN patches in PBS with different glucose concentrations. This result demonstrated that the crosslinked mMN array remained intact after reaching maximum swelling with porous structures. Due to the superhydrophilicity nature of the HA-based hydrogel, increasing glucose concentration brought heterogeneity to the hydrogel and led to a granule-like porous structure internally. It is interesting that the matrix hydrogel became super-absorbent when swollen in a higher concentration of glucose solution (400 mg dL⁻¹). One possible explanation is that the affinity of PBA binds to diol-containing molecules like glucose enhanced the electrostatic repulsion due to increased density of negative charge. The repulsion force between negatively charged HA and anionic form of PBA increased the hydrophilicity of the hydrogel, leading to intensified swelling.

### In Vitro Insulin Release Study

### Method:

To verify even distribution of loaded insulin in the mMN, fluorescein isothiocyanate-labeled insulin (FITC-insulin, 0.1 mg) was loaded into the patch, see FIG. 5A. Glucose-responsive insulin release from mMN patches was determined by incubating the mMN patch in release media (5 mL) at 34° C., see FIG. 5B. The release media contains PBS buffer (pH 7.4) and varying glucose concentrations (50, 100, or 400 mg dL-1). Media containing released insulin was quantified by a Pierce™ Coomassie plus protein assay at 595 nm using a BioRad UV-vis plate reader. The amount of released insulin was calibrated with an insulin standard curve. The pulsatile release profile of the mMN patches was analyzed by incubating mMN patches in release media containing 400 mg dL⁻¹ glucose for 30 min, see FIG. 5C. The media was then removed and replaced with fresh media containing 100 mg dL⁻¹ glucose for another 30 min. This cycle was repeated for 3 hr, and the released insulin was measured using the same protein assay described earlier.

### Result:

The mMN system showed 100% insulin loading efficiency and a high loading capacity (18.2 wt %) owing to the direct addition of insulin during mMN formation, providing an advantage for multi-cycle insulin delivery. The rate and extent of insulin release from the mMN at hyperglycemic state (200 and 400 mg dL-1) were greater than those at euglycemic (100 mg dL-1) and hypoglycemic (50 mg dL-1) levels, demonstrating the mMN patch delivers insulin in a glucose-responsive manner. As the glucose concentration increases, more glucose molecules replace catechol groups in the catechol-AFBA complex, forming more glucose-AFBA complex and causing more mMN swelling and insulin release. Conversely, when the glucose concentration decreases in the media, the driving force for catechol-AFBA complexation increases, thereby regenerating the secondary crosslink network while lowering the rate of insulin release in a feedback control loop. As a result, a pulsatile insulin release profile was observed when the patch was immersed alternatively in the euglycemic and hyperglycemic solutions every 30 min for several cycles. This cyclic high-low insulin release in response to glucose concentrations affords application of the mMN for daily glycemic management.

Secondary Structure of Insulin Released from the mMN Patch

### Method:

The secondary structure of insulin was determined by far-UV circular dichroism (CD) spectropolarimetry. CD spectropolarimetry in the at the far-UV wavelength region was used to characterize any protein secondary structure deviations of insulin. Insulin released from mMN was collected and measured using a spectropolarimeter (Jasco J-810, MD, USA) equipped with a Peltier temperature controller set to 25° C. Samples were filtered and diluted and transferred to a 1 cm path length quartz cuvette for far-UV measurements. Samples were scanned at 1 nm intervals between 200 and 260 nm using an 8 sec response time. The measurement was repeated three times and averaged.

### Results:

The CD spectra of freshly released insulin from the mMN patch showed two signature bands at 208 nm and 222 nm, predominantly attributed to the alpha-helices of insulin (FIG. 5C), which were unchanged as compared to fresh insulin.

Molecular Interaction of Polymer with Insulin by Molecular Modeling

Alongside with experimental phase, molecular modeling methods and computational biology were employed as well. To identify the possible stabilization effects of the polymer on the insulin molecule, the interaction mode between peptide hormone and the functionalized polymers was investigated by all-atom molecular dynamics (MD) simulations. MD is an important and valuable approach for investigating the particle location in space. This approach replaces a single-point model with a dynamic model that forces the nuclear system to move. In MD, the numerical solution of the classical Newtonian dynamic equations is used to realize the simulation of motion.

For simulating the effect of HA-functionalized polymer on the stability of insulin, a hormone molecule with the PDB ID of 4INS was downloaded from the protein data bank. Overall, two independent systems (in the absence or the presence of the polymer) were introduced to the MD studies by Desmond package from Schrödinger Inc. For simulating a 200-disaccharides polymer, 10 chains of HA, each containing 20 disaccharide units were constructed. The disaccharide units were HA (76%), HA-DA (16%), and HA-AFBA (8%), which were randomly integrated into each chain. Both systems, free insulin or in complex with the polymer, were solvated in explicit TIP3P (Three-site transferrable Intermolecular Potential) water model and the OPLS3 (Optimized Potentials for Liquid Simulations version 3) force field parameters.

Temperature of 310 K, pH of 7.4, and a pressure of 1 bar with the simulation length of 100 ns were assigned for each MD run. The particle-mesh Ewald method was employed to calculate the long-range electrostatic interactions. The cut-off radius for computing the Coulomb interactions was 9.0 Å and a cubic periodic box with periodic boundary conditions was defined for both systems in the solvation step. For neutralizing each system, Na+ and Cl− counter ions were added. The distance of 10.0 Å was assigned between the periodic boundary conditions and the closest free insulin or insulin/polymer atom. For each system, the Martyna-Tuckerman-Klein chain coupling scheme and Nose-Hoover chain coupling scheme were engaged for the pressure and temperature control during MD simulation, respectively. A total of 1000 frames/MD runs were allotted, and the trajectories were saved at 10 ps intervals for further analysis.

The interaction mode between insulin and the functionalized polymers was studied using all-atom molecular dynamics (MD) simulations for a mixture where the molar ratio of polymer chains to insulin was assigned according to the ratio used experimentally. FIG. 6A shows the snapshots of insulin at t=0 (starting point), 100 ns, 200 ns, 300 ns, 400 ns, and 500 ns in the presence of HA-DA and HA-AFBA polymers.

As shown in FIG. 6A, the polymer chains surrounded the insulin molecule and over time, they completely encapsulate the insulin molecule. To understand the effects of the polymer on the structure and stability of the protein, the root mean squared deviation (RMSD) values of insulin in the absence or presence of the polymers were analyzed. see FIG. 6B. In this study, the RMSD values of the polypeptide alpha carbon (Ca) atoms were calculated during the simulation with reference to its initial structure. At the beginning of the simulation (t=0 to t<150 ns), the RMSD values of insulin in the presence of polymers were higher than its free state. The higher mobility of the peptide can be attributed to the initial movement of the molecule to interact with the polymer residues. However, at 150 ns, the RMSD decreased, attributable to the stabilized interaction of insulin with the polymer, FIG. 6B.

The root mean square fluctuation (RMSF) of each residue in the insulin molecule was also monitored to determine the effects of polymer on the dynamic behavior of polypeptide residues, see FIG. 6C. The computed RMSF values for the Ca atoms in insulin were compared to the B-factor (thermal factor) of polypeptide atoms extracted from X-ray crystallography experiments. A similar trend observed for computed RMSF and B-factor values validated our MD simulation results. As depicted, all insulin residues are sensitive to the presence of the polymers, as shown by their lower RMSF values in the complex state. This characteristic can be attributed to the favorable interactions between insulin and the functionalized-HA polymers.

FIG. 6D exhibits typical results for the radius of gyration (Rg) of insulin in the absence and presence of the polymers. It is seen that the protein in the presence of the polymer is nearly unaltered at 300 ns and onwards as compared to free insulin, suggesting that the polymer has no remarkable effect on the Rg of insulin. Previous studies have demonstrated that hydrogen bonds (H-bonds) have a pivotal role in the complex formation between polymer and protein molecules. Thus, the intra-H-bonds in insulin molecule and inter-H-bonds between insulin and the polymers were studied. The average number of intra-H-bonds in both free and bound insulin remained unchanged at an average value of 38, see FIG. 6E, while the average inter-H-bonds between insulin and the polymer during the simulation were found to be 12.3, see FIG. 6F.

This hydrogen bonding pattern may be explained as that the protein is stabilized by HA-based polymers that shield the protein. Residues Ile2 (chain A), Cys7 (chain B), Thr8 (chain A), Ser12 (chain A), Glu13 (chain B), Leu17 (chain B), Tyr19 (chain A), Asn21 (chain A), Pro28 (chain B), Lys29 (chain B), and Ala30 (chain B) from insulin participate in H-bonding with the functionalized-HA polymers. It has been found that Asn21, as a conserved residue, plays a key role in the deamination of the protein in an acidic medium, and in maintaining the specific spatial configuration of insulin required for its bioactivities. Because Asn21 residue is located at the end of the chain, its H-bonding with the polymer may stabilize insulin structure and maintain insulin bioactivity.

On the other hand, H-bonding of backbone residues Leu17 (chain B) and Ile2 (chain A) with the polymer could block insulin fibrillation, because the point mutation of Leu17 (chain B) could delay the lag phase of insulin fibrillation, and Ile2 (chain A), a hydrophobic residue, acts as a nucleation-prone residue in insulin fibrillation. In addition to hydrogen bonding, two major types of free energy of binding (electrostatic interaction and van der Waals) and total binding energy between insulin and the polymer were also computed.

As shown in FIG. 6G, electrostatic interaction between insulin and the polymer is more significant than van der Waals' interactions. The average total binding energy of insulin with the polymers is −258.2 kcal mol⁻¹ (FIG. 6H). FIG. 6I shows that the average solvent accessible surface area (SASA) of insulin is reduced from 3674.15 Å2 for free insulin to 2319.33 Å2 for insulin with the polymers, which can be ascribed to the “shielding effect” of the HA-based polymers. As discussed above and reported previously, the high hydrophilic nature of the polymer could provide a stabilization effect, and the shielding effect via encapsulation by ionically charged HA could prevent insulin aggregation. The result suggests that the functionalized-HA polymers can possibly prevent insulin fibrillation and proteolysis. In addition to protein fibrillation, random interactions with water molecules also cause insulin deactivation. Our simulation results showed that the density of radial distribution function (RDF) of water molecules around the insulin was reduced in the presence of the polymers as compared to free insulin (FIG. 6J).

In other words, in the presence of the polymers, more water molecules are excluded from the surface of the insulin, which can decrease the possibility of random interaction of insulin with water molecules. However, the height of the first peak of RDF at the distance smaller than 3 Å related to the first layer of bound water molecules shows the same value for free insulin and polymer-insulin (FIG. 6J).

### Evaluation of In Vivo Efficacy

### Method:

Fasted diabetic rats were treated with either insulin-loaded mMN (58 IU), sham patch (as a control), or s.c. insulin injection (0.5 U kg⁻¹). Rat dorsal skin was shaved, treated with hair removal cream, and dried prior to patch applications. Blood glucose was monitored using tail-pricking method every 5-15 minutes.

### Result:

To investigate the in vivo efficacy of the mMN patches against hyperglycemia, streptozotocin (STZ)-induced T1D male Sprague-Dawley rats were randomly grouped and treated with either insulin-loaded mMN patch (58 IU patch⁻¹), sham patches (as a control), or s.c. insulin injection (0.5 U kg⁻¹). All T1D diabetic rats were fasted for 5 hr before the study began. The mMN patches, sham patches or insulin injections were administered on the rats at t=0, and their BGLs were monitored for 8 hr. As shown in FIG. 7, the BGLs in the control groups treated with sham patches remained at hyperglycemia throughout the study. However, the BGLs of the rats treated with mMN patch and insulin injections rapidly decreased and fell below 165 mg dL⁻¹ within 1 hr. The rats treated with insulin injection did not remain in the normoglycemic state and their BGLs gradually increased to hyperglycemia after 3.5 hr. Conversely, the mMN patch successfully regulated BGLs of the rats within a tight glycemic target range (<190 mg dL⁻¹ and >70 mg dL⁻¹) for more than 7 hr, which is longer than matrix MN patches reported by other research groups.

### Evaluation of In Vivo Efficacy (Glucose Challenge Study)

To study the glucose regulation capability of the mMN patch, intraperitoneal glucose tolerance tests (IPGTT) were conducted by injection of 2 g kg⁻¹ of glucose 2 hr-post mMN application or insulin injection to lower the initial plasma glucose level to a similar normoglycemic level. As illustrated in FIG. 8, the BGLs in the T1D rats treated with mMN returned to normoglycemic state after the glucose levels peak similar to the healthy rats. In contrast, the T1D rats 2 hr after subcutaneous (s.c.) insulin injection experienced sustained hyperglycemia after the glucose challenge. The glucose area under the curve (AUC), a measure of sustained glucose exposure for 120 min, in the s.c. insulin-treated rats was 5-fold that in the mMN patch-treated rats (FIG. 9). The glucose regulatory effect of the mMN patch can be ascribed to its glucose-dependent insulin release.

As shown in FIG. 10, at t=0 (i.e., 2 hr-post mMN application) when the BGL returned to euglycemia, the plasma insulin in the mMN patch-treated rats was at a low level like that in the sham patch-treated group, indicating undetectable insulin was released from the insulin mMN patch at this point. After the glucose challenge at t=0, the plasma insulin level increased dramatically by up to 9.8-fold at 30 min and 60 min and then dropped to the basal level again at 120 min, while no change was found in the sham patch-treated rats (FIG. 10). These results indicate that the mMN patch is able to adjust the insulin release rate according to blood glucose levels, thus maintaining euglycemia for an extended time without causing hypoglycemia and rapidly suppressing hyperglycemia. Owing to the limitation of the duration of fasting the T1D rats and the number of blood samples that can be taken from each rat in a continued study, the longer-term efficacy of the insulin mMN patch will be evaluated using a large animal model, which is being pursued by our group.

### Biocompatibility of the Polymers and mMN

### Method:

The in vitro cytotoxicity study of the mMN patch was measured by conducting 3-(4,5)-dimethylthiahiazo(-z-y1)-3,5-di phenytetrazoliumromide (MTT) assay on both NIH/3T3 fibroblast cells and HaCaT Human keratinocyte cells to mimic skin and tissue. Briefly, both cell types were seeded on tissue-treated 96-well plates at 10,000 cells per well. After 24 hr incubation in Dulbecco's Modified Eagle Medium (200 μL) with 10% fetal bovine growth serum, cells were treated with samples and incubated for 24 hr at 37° C. After that, MTT reagent (100 μL) was added to each treated well and incubated for 4 hr, followed by adding 10% SDS in 0.01 M HCl (100 μL) to each well. After incubating for another 4 hr, the absorbance of the plate was read at 570 nm using a BioRad UV-vis plate reader. The percent survival was determined using the following equation and plotted on a semi-log scale.

$$\% survival = \frac{sample sigmal - background signal}{control sigmal - background signal} \times 100$$

The in vitro cytotoxicity of the patch materials was evaluated on mouse fibroblast cells (NIH-3T3) and human keratinocyte cells (HaCaT) using MTT assay. As shown in FIG. 11, due to the high biocompatibility of the HA-based polymers, no noticeable toxicity was observed.

### Local Tissue Biocompatibility by Histology Staining Study

### Method:

The mMN patch was applied to shaved T1D rat skin for 12 hr. After day 1, day 3, or day 7 post-patch removals, the treated skin tissue and healthy skin without patch treatment were harvested and fixed in 10% buffered formalin for 24 hours, embedded in paraffin and sectioned into 5 mm thick slices. Then, the samples were stained with H&E, CD 68, and Masson's Trichrome (MTC).

### Results

To further ensure the safety of our patch, in vivo biocompatibility studies were also examined after the mMN patch application on rat skin for 1, 3, and 7 days by analyzing hematoxylin & eosin (H&E), CD68, and Masson's Trichrome (MTC) staining (FIG. 11). From the H&E staining results, slight inflammatory neutrophil infiltration was observed 12 hr after mMN application on day 1, which was recovered on day 7. CD68 staining results showed the presence of macrophages at the site of perforation of rat skin treated with mMN patch were minimal by day 7, which was comparable with healthy rats without treatment, see FIG. 12. Lastly, MTC staining results appeared similar between the treatment and control groups, demonstrating no significant changes to the collagen and extracellular matrix structure of the perforated rat skin across 7 days after patch application. Overall, histological analysis across all types of staining indicated minimal evidence of cell inflammation, signs of tissue damage, and skin structural deformation post-patch applications.

The specific embodiments described above have been shown by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

### Section B

### “Smart” Glucagon-Delivering Matrix MN Patch Using Self-Crosslinkable Catechol-Containing Polymers

Dynamic metal-ligand complexation has attracted significant interest in both fundamental and applied material science. The dynamic binding interactions could instantly dissociate, exchange, and reform. Therefore, they can be used in designing “smart” materials with reversible “ON/OFF” bindings. Currently, hydrogels formed by catechol-mediated reversible binding are under development for wide range of applications. Catechols are known to form reversible complexes with metal ions, metalloproteins, and boron-containing molecules with the binding affinity being dependent on the nature of the molecules. For instance, the catechol groups exhibit high specific affinities for iron, copper, cobalt, and relatively lower affinity for calcium and zinc. Catechol-metal complexation is reversible and can be broken in the presence of a molecule with a higher affinity binding constant. Boronic acid-containing monomers are an additional class of molecule, which can form complexes with catechol groups. However, these interactions can be influenced by glucose molecules in a competitive manner.

At high glucose concentration, boronic acids react with glucose molecules to generate cyclic boronate esters. As the glucose concentration decreases, the boronic acid derivatives dissociate and bind competitively with catechol groups. In this scenario, if the catechol groups pre-chelated with a metal-containing molecule that has a lower affinity binding constant than boronic acid, the boronic acid will replace the metal-containing compound. It is also known that glucagon has inherent properties for interaction with metal ions (e.g., zinc). Previously, zinc-glucagon (Z-GCN) was successfully used in the management of a male infant with persistent idiopathic hypoglycemia, and children with idiopathic infantile hypoglycemia. Interestingly, it has been found Z-GCN induces more prolonged effects than native glucagon in maintaining euglycemia in insulin-treated people with type 1 diabetes.

In this work, we proposed to use the catechol-boronic acid based-chemistry to design a closed-loop self-crosslinked hydrogel-based MN patch using dopamine and 4-amino-3-fluorophenylboronic acid (AFBA)-functionalized HA-based polymers. These co-polymers can deliver a blood glucose-raising agent (Z-GCN) at low glucose level to prevent the onset of hypoglycemia (FIG. 13). In this design, at high glucose concentrations, the AFBA functional groups reversibly bind with glucose to generate cyclic boronate esters. Meanwhile, the catechol functional groups of dopamine (DA) bind to Z-GCN through a metal-ligand complexation. However, as the glucose concentration decreases, the AFBA will be favored over Z-GCN in binding with DA, due to its higher binding affinity and its free energy value, as confirmed by molecular dynamics (MD) simulations and quantum mechanics (QM) computations, which promotes the release of Z-GCN at low glucose concentration. It is well known that many proteins/peptides have a high binding affinity to metal ions almost half of all known proteins require metal ions to maintain their structures and biological functions. Hence, we believe that the introduced mechanism driven by the competitive binding of catechol-metal ions, is of great implication in drug delivery applications for various protein/peptides.

This is the first demonstration of a self-crosslinkable and glucose-responsive matrix microneedle system fabricated from a simple and mild crosslinking method to regulate blood glucose levels automatically by delivering hormones glucose-responsively. Although microneedle patches have been published and patented for diabetes management (Web of Science: 85), the amount of knowledge for using self-crosslinkable, glucose-responsive, and matrix-based microneedle patches for diabetic management cannot be found in literature. Only a handful of matrix-based microneedle patches have been reported for glucose control in diabetes (Web of Science: 10). However, most prior published works used thermal or ultra-violet irradiation containing organic solvents, chemical crosslinkers, and thermal/photoinitiators to fabricate matrix MN patches. Moreover, the conjugation of AFBA as the glucose-responsive component in matrix microneedles has not yet been reported.

Furthermore, regarding simple methods for fabricating microneedle systems, only two technologies have been reported. In the first, (1) Gu et al (2019) (US Provisional Patent Application No. 617/270,953) have developed a method to fabricate glucose-responsive matrix microneedle patch in situ for insulin delivery. However, the system was formed via in situ polymerization and crosslinked in organic solvent using a conventional method that requires ultraviolet irradiation for 30 minutes. Such a method created excessive unreacted residues (e.g., monomers, crosslinkers and photoinitiators) left behind, causing toxic effects when entering the body after patch administration. Even though such a system was immersed in phosphate buffered saline containing 20% ethanol solution for purification, the system still had a tracible amount of unreacted monomers that were detected. Unwanted drug release (~10%) also occurred during the purification step. Moreover, the bioactivity of loaded protein may be reduced by such polymerization conditions.

In the second (2) Chen et al (2021) recently reported a mild preparation method was developed to fabricate glucose-responsive microneedle patch for insulin delivery. Such a system utilized the borate ester bond with polyvinyl alcohol to form a pre-hydrogel solution without polymerization and organic solvents. However, the absence of covalent crosslink of such system did not truly achieve glucose-dependent drug release in vitro and lack in vivo evidence for its pulsatile release behavior. Moreover, the pre-hydrogel also needs to be treated with a freeze/thaw process 3 times to make crystalline crosslinking of polyvinyl alcohol chains, which is inconvenient.

As previously mentioned, at the heart of the introduced system is a rationally-designed self-crosslinkable and glucose-responsive hydrogel and successfully formulating this functional polymer required various supporting innovations. Some of the key points are listed below:

Conjugated PBA monomers onto HA polymer with organic solvent had a low conjugation efficiency due to its poor solubility in water. Choosing a suitable water-based solvent was of crucial importance for this system, and a water-ethanol mixture at a specific ratio was found to improve boronate conjugation efficiency.

The ratio between DA and AFBA conjugates in the composition of the HA-based hydrogel was rationally selected for the optimal glucose-responsive release profile while forming a stable non-degradable 3D matrix network to provide clinically relevant drug loading capacity for effective prolonged glycemic control.

The numbers of AFBA conjugates can drastically change the hydrogel characteristics. For instance, it was found that low AFBA conjugation ratio had unstable gelation outcomes, weakening the integrity of matrix MN patches. Too high or low degree of DA conjugation also affects self-crosslinking efficiency and its ability for MN fabrication.

A composition for preventing hypoglycemia: comprising a hydrogel-based MN that self-crosslinked via catechol-metal interaction.

A composition for delivering glucagon at hypoglycemia condition: wherein the crosslinking method does not influence the bioactivity of blood glucose-raising therapeutic agent.

A composition for delivering glucagon at hypoglycemia condition: comprising a hydrogel containing the containing catechol groups and metal ions that competitively bind with glucose causing rapid release of the blood glucose-raising therapeutic agent.

Synthesis of Multifunctional HA Polymer with Dopamine (DA) and AFBA

Catechol- and boronic acid-containing copolymers were prepared by functionalizing HA (MW=300 KDa) with various ratios of DA and AFBA. The chemical structure of HA-DA and HA-AFBA were characterized using ¹H-NMR. The N-acetyl peak of HA appeared at 1.9 ppm. Multiplets from 3 to 3.8 ppm were associated with disaccharide units in the HA backbone. Chemical shift at b=6.7 to 7.32 ppm corresponds to the catechol aromatic ring of dopamine. The degree of substitution of DA was calculated to be 17% using the integral of the peak related to the N-acetyl group of HA at b=1.9-2.0 ppm to the aromatic peaks of dopamine at b=6.7-7.0 ppm. %). The degree of AFBA conjugation was determined to be around 12.9% using the ¹H-NMR spectrum by integration of the aromatic peaks of AFBA at b=7.5-8.0 ppm to the peak at b=1.9-2.0 ppm related to the N-acetyl glucosamine of HA.

Modification of Zinc-Glucagon (Z-GCN) Zinc glucagon is made as described in Tarding et al. (European Journal of Pharmacology 7:206-210 (1969)). In brief, zinc-glucagon was made by suspending freeze-dried glucagon in a zinc acetate buffer, for a final concentration of 1 mg glucagon/ml, 0.05 mg zinc/ml. The secondary structure of the peptide was characterized before and after Zn-coordination/modification by CD spectropolarimetry.

### Fabrication and Characterization of the Self-Crosslinked MN Matrix Patch for Glucose-Responsive Delivery of Glucagon (Z-GCN)

A self-crosslinkable MN patch was successfully fabricated using a rationally selected ratio of HA-DA and HA-AFBA (2:1). First, 1 mL HA-AFBA solution (50 mg/mL) containing 3 mg of glucose was mixed with 1 mL HA-DA solution (50 mg/mL) containing adequate amount of Z-GCN. Next, the pH of the mixture was adjusted to 8 using 1 M NaOH and stirred with a spatula until the color of the solution turned to brown. Then the mixture was added into a polydimethylsiloxane (PDMS) MN mold and vacuumed under 25 mmHg for 5 minutes to remove trapped air. The mold was kept at room temperature in a fume hood overnight. After complete desiccation, the MN patches were carefully separated from the mold and trimmed.

### Chemical Characterization

### UV-Vis Method:

UV-Vis absorption was studied for HADA solution and in the presence of AFBA and Z-GCN with and without glucose. UV-Vis analysis was carried out with a UV-Vis spectrometer using quartz cuvettes (1 cm path length). To obtain higher resolution in the UV-Vis spectrum, AFBA monomer was used instead of HA-AFBA.

### Result:

The UV-Vis spectrum of the pure HA-DA revealed a peak at 280 nm representing the catechol groups of polymers (FIG. 15A). Addition of Z-GCN to HA-DA showed a new peak at ~360 nm indicating the formation of zinc-catechol mono-complexation. Similarly, addition of AFBA to HA-DA polymer solution showed a new peak around 470 nm related to the formation of boron-catechol complexation. However, when both Z-GCN and AFBA were added to the HA-DA solution, the UV-Vis spectra only revealed a peak related to the AFBA-catechol binding, confirming that the interaction between catechol-boron is more pronounced than catechol-Z-GCN. The opposite trend was seen for this sample in the presence of glucose (70 mg/dL), where only the peak related to the interaction between Z-GCN and catechol could be observed, indicating that in the presence of glucose, glucose will be favored over catechol in binding with boronic acid.

### Gamma-Ray Sterilization of MN Patch

The MN patches were sterilized using a gamma-ray irradiation facility at the University of Toronto to study the effect of sterilization on the mechanical properties of the patches. The equipment used for the gamma irradiation is the Gamma Cell, type G.C. 220. The Gamma Cell utilized an annular Co-60 source enclosed within a lead shield chamber. The MNs were exposed to a total dose of 25 kGy in a sealed chamber with evenly distributed gamma field for one week to render them sterile. The sterilized MNs were observed under microscope to check for the changes in the morphological characteristics.

### Mechanical/Adhesive Property:

### Method:

The mechanical strength of MN arrays was tested using Instron 3366 universal testing machine equipped with a compression cell. The MN arrays were placed vertically on a compression plate. The distance between two plates was set to 2 mm. A compressive force at a speed of 0.5 mm/min was applied. The compression threshold was set to 10 N. The load (force; N) and displacement (distance; mm) were recorded every 0.05 s to create the load-displacement curve. For measurement of hydrogel adhesion, lap shear joints were prepared, following a process frequently used for measuring the adhesive strength of hydrogel-based tissue adhesives. Two ribbons of hydrogels (length l×width w=75 mm×25 mm) were cut. They were brought into contact with two glass slides coated with a medical tape (melt blown polyurethane) to mimic skin like texture. The lap joint was slightly pressurized with a 20 g weight for 5 minutes then the two ends of the glass were clamped to the tensile machine. The shear adhesive test was performed at a shear velocity of 100 mm min⁻¹ under ambient condition. The applied force and displacement were recorded.

### Result:

The mechanical compression testing of the MN patch demonstrated a force-at-break of 0.45 N per needle (FIG. 14C) which has sufficient strength for penetration into the rat's skin. The mechanical property of MNs were not significantly affected by gamma irradiation (25 kGy) and the sterilized MNs retained native sharpness. The conjugation of DA to HA was also found to significantly increase the tissue adhesiveness of the patch. As shown in FIG. 14F, the adhesive stress of the hydrogel was significantly greater than non-catecholic hydrogels. It should be mentioned that MNs swelling during the application of hydrogel-based MN patches may weaken the adhesiveness of the hydrogels. However, the strong adhesive property of catechol groups under wet condition provided a beneficial adhesion property to this patch. This is due to the interactions of the catechol groups with the thiol or amine groups of the skin surface. It should be mentioned that MNs swelling during the application of hydrogel-based MN patches may weaken the adhesiveness of the hydrogels. However, the strong adhesive property of catechol groups under wet condition provided a beneficial adhesion property to this patch. This is due to the interactions of the catechol groups with the thiol or amine groups of the skin surface.

### Swelling Studies:

### Method:

The swelling profile of the MN patch was assessed by immersing the patch in pH 7.4 PBS. The MN patch swelled rapidly upon immersing in PBS buffer. The net weight of the swelled MN patch was carefully measured at predetermined time points. The swelling ratio was calculated based on a formula of the weight of the MN patches at various time (Wₜ) to its initial net weight (W₀) as shown below.

$$Swelling Ratio = \frac{{W}_{t} - {W}_{0}}{{W}_{0}} ⋆ 100$$

### Result:

The swelling ratio of the patch was assessed by immersing the patches in pH 7.4 phosphate buffered saline (PBS) (FIG. 14E). The MN patch exhibited a rapid super swelling behaviour due to its porous structure with maximum swelling occurring at ~60 minutes.

### In Vitro Release Study

### Method:

The secondary structure of modified glucagon (Z-GCN) was determined by far-UV circular dichroism (CD) spectropolarimetry. Glucose-responsive Z-GCN release from MN patches was determined in PBS buffer (pH 7.4) containing glucagon solubilizer (MSB) and varying glucose concentrations (50, 100, or 400 mg/dL). Vials were placed in a 34° C. incubator to mimic skin temperature. Drug released overtime was measured using RP-HPLC. The pulsatile release profile of the MN patches was also analyzed by replacing the released media with different glucose concentration.

### Results:

The glucose-responsive release of Z-GCN from the MN patch was determined in PBS buffer containing different glucose levels. Approximately 52% and 31% of the loaded Z-GCN was released after 40 min in media containing 50 and 70 mg/dL of glucose, respectively (FIG. 16A). Compared with the release rate of Z-CGN at hypoglycemic state, the preloaded Z-GCN showed significantly slower release at hyperglycemia with about 5% of the loaded Z-CGN released to the media. Such a minimal drug release indicates that the Z-GCN was tightly bound to the catechol groups and its delivery profile is independent of volume changes of the MN that occurs during the swelling. The repeated “ON/OFF” pulsatile release profile of Z-GCN from the patch was also carried out by immersing Z-GCN-loaded MNs at different glucose concentrations (FIG. 16B).

The results indicated a cyclic profile with a controllable Z-GCN release at various glucose concentrations, making it a safe and controllable drug delivery system. Far-UV CD spectropolarimetry was used to evaluate the stability of the Z-GCN and released Z-GCN from the MN patch, and the result was compared to native glucagon (FIG. 16C). The CD spectra of Z-GCN showed two similar peaks with native glucagon, which is predominantly attributed to the alpha-helix of this peptide. The CD spectra of released Z-GCN from MN patch remained the same as freshly prepared glucagon, suggesting that the secondary structure of glucagon was preserved during zinc coordination, MN fabrication, and release study. To better understand the change in secondary structure fractions, spectra deconvolution was performed using the SELCON3 algorithm from the CDPro software. From deconvolution, it was found that fresh glucagon had 58.9% alpha-helical content, (FIG. 16D). Z-GCN and released Z-GCN retained 57.8 and 54.2% alpha-helical content respectively. FIG. 16E depicts the interaction mode between Z-GCN and the polymer, revealing a multifaceted network of molecular associations. Within this complex, the guanidinium group of Arg 18 from Z-GCN forms two hydrogen bonds with the carbonyl moiety of HA-AFBA, contributing to the stability of the complex.

Concurrently, Tyr 10 establishes a hydrogen bond with the HA moiety of the polymer, enhancing the specific interaction between Z-GCN and the polymer. Additionally, the imidazole ring of His 1 from Z-GCN engages in interactions with two HA residues from the polymer, while the amino group of His 1 further creates a hydrogen bond with the polymer's HA (FIG. 16E). These intricate interactions collectively illustrate the structural basis underlying the association between Z-GCN and the polymer, offering insight into their potential functional interplay.

### Quantum Mechanics (QM) Computations

### Method:

The zinc-catechol, glucose-AFBA, zinc ion, glucose, and AFBA-catechol, and zinc-Phe-zinc-catechol were constructed with GaussView software. For calculating the reaction energies of reactants and products, density functional theory of B3LYP-D3 and basis set of LACVP** were used. In all QM studies, water was selected as the solvent. Due to the presence of zinc, the geometries of the reactants and products were optimized with PCM (polarizable continuum model) and followed the single point energy calculation by PBF (Poisson Boltzmann Finite element) model. All QM calculations were conducted using Jaguar.

### Results:

Quantum mechanics (QM) modeling was further employed to elucidate possible replacement of Zn-GCN in the Zn-GCN-catechol complex by boronic acid (AFBA) at a low concentration of glucose. The thermodynamic analysis showed that AFBA-catechol is the favored primary product, with a Gibbs free energy of −518.23 kcal/mol. Radial distribution function (RDF) data confirmed a strong interaction between the zinc and residue Phe 6 of GCN in a 100 ns MD simulation (FIG. 15D). Phe-Z-catechol was used as a surrogate model for Z-GCN, replacing Zn-catechol in the second QM simulation run (FIG. 15E). This change supported earlier results, confirming the primary product's thermodynamic favoring with a Gibbs free energy of −600.67 kcal/mol. Cluster analysis of MD trajectories further disclosed the interaction mode between the zinc ion, GCN, and polymer. As shown in FIG. 15F, the zinc ion coordinates with both HA from the polymer and GCN's residue Phe 6 and interacts with three water molecules. The key role of Phe 6 in receptor recognition is known. Density functional theory was used to study the catechol-AFBA complex formation, focusing on the highest occupied molecular orbital (HOMO), the lowest unoccupied molecular orbital (LUMO), and energy gap (ΔE) (FIG. 15G). In the examined reaction, the Phe Zn in the Phe Zn-catechol complex is replaced by AFBA, leading to the formation of AFBA-bound catechol. The process is characterized by a HOMO energy of −0.173495 eV and a LUMO energy of −0.065175 eV. This indicates that Phe Zn-bound catechol acts as an electrophile, with increased reactivity due to a lower energy gap (0.10832 eV). Conversely, glucose-bound AFBA, with HOMO and LUMO energies of −0.187550 eV and 0.009338 eV, respectively, serves as a nucleophile. The reaction progresses through a nucleophilic attack by glucose-bound AFBA on Phe Zn-bound catechol, causing the displacement of the Zn-bound Phe and releasing glucose. AFBA then binds to catechol to produce the thermodynamically favored product, catechol-bound AFBA. This comprehensive analysis, grounded in HOMO and LUMO energetics, unveils the intricate molecular interactions that drive this transformation. It not only validates the thermodynamic feasibility of forming catechol-bound AFBA but also emphasizes the orchestrated interplay between nucleophilic and electrophilic behaviors in the reactants. Such understanding augments our knowledge of this complex reaction pathway and potentially opens avenues for further exploration and application.

### Molecular Modeling

Alongside with experimental phase, molecular modeling methods and computational biology were employed as well. To identify the possible stabilization effects of the polymer on the glucagon molecule, the interaction mode between peptide hormone and the functionalized polymers was investigated by all-atom molecular dynamics (MD) simulations. MD is an important and valuable approach for investigating the particle location in space. This approach replaces a single-point model with a dynamic model that forces the nuclear system to move. In MD, the numerical solution of the classical Newtonian dynamic equations is used to realize the simulation of motion.

### Method:

For simulating the effect of HA-functionalized polymer on the stability of glucagon, a hormone molecule with the PDB ID of 1GCN was downloaded from the protein data bank. Overall, two independent systems (glucagon in the absence or the presence of the polymer) were introduced to the MD studies by Desmond package from Schrödinger Inc. Based on the ratio extracted from NMR measurement, the molar ratio of disaccharides of HA, HA-DA, and hyaluronic acid functionalized by HA-AFBA were set to 78%, 16%, and 8%, respectively to construct a crosslinked polymer. Both systems, free glucagon or in complex with the polymer, were solvated in explicit TIP3P water model and the OPLS3 force field parameters. A temperature of 310 K, pH of 7.4, and a pressure of 1 bar with the simulation length of 100 ns were assigned for each MD run. The particle-mesh Ewald method was employed to calculate the long-range electrostatic interactions. The cut-off radius for computing the Coulomb interactions was 9.0 Å and a cubic periodic box with periodic boundary conditions was defined for both systems in the solvation step. For neutralizing each system, Na⁺ and Cl⁻ counter ions were added. The distance of 10.0 Å was assigned between the periodic boundary conditions and the closest free glucagon or glucagon/polymer atom. For each system, the Martyna-Tuckerman-Klein chain coupling scheme and Nose-Hoover chain coupling scheme were engaged for the pressure and temperature control during MD simulation, respectively. A total of 1000 frames/MD runs were allotted, and the trajectories were saved at 10 ps intervals. After equilibrating each system, replica exchange molecular dynamics (REST) was conducted. For enhanced conformation sampling using REST, total of 10 replicas were generated to be simultaneously simulated for 100 ns each at temperatures ranging from 300 K to 481 Kln REST, the total interaction energy of the system was decomposed into three components: the protein/polymer intramolecular energy, Eₚₚ; the interaction energy between the protein/polymer and water, E_{pw}; and the self-interaction energy between water molecules, E_{ww}. Replicas running at different temperatures then evolve through different Hamiltonians involving relative scaling of these three components. To be specific, the replica running at temperature Tₘ has the following potential energy:

$${E}_{m}^{R E S T} ( X ) = {E}_{p p} ( X ) + \frac{{β}_{0} + {β}_{m}}{2 {β}_{m}} {E}_{pw} ( X ) + \frac{{β}_{0}}{{β}_{m}} {E}_{w w} ( X )$$

Here, X indicates the configuration of the whole system, βm=1/kBTm, and T0 is the favorite temperature. The potential for replica running at T0 reduces to the normal potential. Imposing the detailed balance condition, the acceptance ratio for the exchange between two replicas m and n depends on the following energy difference:

$${Δ}_{m n} ( REST ) = ( {β}_{m} - {β}_{n} ) [ ( {E}_{p p} ( {X}_{m} ) + \frac{1}{2} {E}_{p w} ( {X}_{n} ) ) - ( {E}_{p p} ( {X}_{m} ) + \frac{1}{2} {E}_{p w} ( Xm ) ]$$

The water self-interaction energy, E_{ww}, does not appear in the acceptance ratio formula, and this is the reason only a relatively small number of replicas are sufficient to achieve good exchange probabilities in REST.

### Results:

The REST simulation in this study methodically explored interactions between Z-GCN and polymers and the polymer's stabilizing mechanism on glucagon's native structure. Timelines for all 10 replicas, revealed temperature-dependent changes in GCN's secondary structure and the polymer's modulatory role. Specifically, increasing the temperature from 300 to 481 K led to a reduction in the α-helix content of free GCN from 56.01% to 21.79%. This change indicates a temperature-induced perturbation in the peptide's conformation, reflecting the complexity of thermal-responsiveness within its structure and emphasizing the polymer's role in this dynamic process.

Temporal snapshots of the Z-GCN both in the absence and presence of a polymer revealed significant structural insights. Without the polymer, Z-GCN's helical structure distorts overtime, indicating instability. In contrast, the polymer's presence remarkably preserves the α-helical structure thanks to interactions between the two (FIG. 16E). An analysis of the root mean squared deviation (RMSD) values shows increased stability in the peptide-polymer complex, with the average RMSD of Z-GCN attenuated from 5.57 to 3.33 Å, indicating the increased stability in peptide-polymer complex (FIG. 17A). The root mean square fluctuation (RMSF) of Z-GCN residues was also lowered in the complex state (FIG. 17B), signifying sensitivity to the polymer's presence. Further, hydrogen bonds were computed, reflecting a robust interplay between the polymer and Z-GCN (FIG. 17D), but without remarkable changes in the intra H-bonding within glucagon (FIG. 17C). The radial distribution function (RDF) was used to analyze the spatial arrangement of water molecules around Z-GCN, revealing a pronounced shift in the hydration shell due to the polymer's influence (FIG. 17E). The RDF plot also illustrates a distinct pattern between Z-GCN and specific polymer residues, signifying preferential proximity between Z-GCN and the HA-DA residue (FIG. 17F). This suggests its role in influencing Z-GCN's orientation and stability within the polymer complex. The binding energy analysis reveals the predominance of electrostatic interactions, significantly overshadowing Van der Waals forces, in the overall binding affinity (FIG. 17G). These insights help to understand the specific manner of Z-GCN's engagement with the polymer (FIG. 17H). Finally, the solvent accessible surface area (SASA) analysis shows a marked decrease in Z-GCN's SASA in the presence of the polymer (FIG. 17I). This reduction may reflect a conformational change or shielding effect by the polymer, altering Z-GCN's interaction dynamics with its surroundings. These comprehensive observations emphasize the critical role of the polymer in stabilizing the Z-GCN structure and provide an understanding of the complex molecular interplay between them.

### Local (Skin) Biocompatibility Study

### Method:

To evaluate the local biocompatibility of the MN patch, the patch was applied onto the shaven dorsal region of STZ-induced T1D rats overnight (12 hours) and removed. The patch-treated skin area was excised one-, three-, and seven-days post-patch removal. The excised tissues were immediately fixed in 10% buffered formalin for 48 hours and transferred to 70% ethanol. The tissues were then sectioned and stained using hematoxylin & eosin (H&E), Masson's trichrome (MTC), and CD68 antibodies. The stained sections were analyzed under high resolution bright-field microscopy to observe for inflammatory markers and tissue damage. H&E stained nuclei of cells purple and extracellular matrix/cytoplasm pink. MTC is a stain for differentiating between cells and connective tissue—muscle fibres, keratin, and cytoplasm are stained red; nuclei are stained blue; and collagen are stained blue/green. CD68 stained for inflammatory markers such as monocytes/macrophages with a dark brown hue.

### Result:

Prior to the in vivo efficacy study, the biocompatibility and safety performance of MN patch were fully evaluated. First, the in vitro cytotoxicity of the MN patch was measured by performing MTT assay on NIH-3T3 fibroblast cells and HaCaT cells (FIG. 18A) (to mimic skin tissue). The results showed that the cell viability was not significantly influenced due to the high biocompatibility of the materials. The amounts of leachable and unreacted compounds from the patch were measured by high performance liquid chromatography (HPLC) (FIG. 18B). After 12 hours of immersion in release media, only 0.0067 mg of DA and 0.002 mg of AFBA were detectable. Furthermore, the local biocompatibility of the MN patch was also evaluated via applying the patch onto the shaven dorsal region of rats and the tissue was analyzed after seven days post-patch application (FIG. 18C). No macroscopic tissue damage was observed across seven days post-patch application. All three stains showed the presence of hair follicles, sebocyte lobules, and other cellular structures that are common to subcutaneous tissue morphology. Minimal presence of inflammatory markers (monocyte/macrophages) were detected with CD68 staining over seven days after patch removal. Overall, the MN patch demonstrated no evidence of inducing tissue damage or inflammatory response and is thus, biocompatible.

### In Vivo Hypoglycemia Prevention (Multiple Insulin Challenge Study)

### Method:

In vivo studies were evaluated in a STZ-induced T1D rat model. The diabetic rats were fasted for 5 hours to ensure the clearance of food from the gut. The rats were then anaesthetized by IP injection of ketamine and shaved and treated with hair removal cream before application of Z-GCN loaded MN patch or sham device (control) at t=0. The insulin challenge was carried out by subcutaneously injecting native insulin at a dose of 3 IU/kg. The blood glucose levels were monitored every 15 minutes for 3 hr with a glucometer using tail pricking/strip-method. Blood samples (250 μL per rat) were collected from the tail vein of rats at predetermined timepoint and centrifuged to isolate plasma followed by storing at −20° C. until assay. Plasma glucagon levels were measured using a human glucagon ELISA kit (R&D System).

### Result:

As shown in FIG. 19A The functionalized glucagon with 5% zinc did not significantly influence the bioactivity of glucagon. The increase in blood glucose after injection of glucagon and Z-GCN were maximal at 30 and 60 min respectively. The duration of action of Z-GCN was found to be higher than glucagon.

After confirmation of its bioavailability, the hypoglycemia prevention capability of the MN patch was tested in vivo following the experimental design shown in FIG. 19B. Rats were initially treated with the Z-GCN-patch or a sham patch (control) and then the insulin overdose injections (3 IU) were given 30 minutes post-patch application.

The blood glucose of rats given the Z-GCN-patch remained well above the hypoglycemic threshold (70 mg/dL) during the experiment (FIG. 19C). In contrast, the blood glucose of rats given sham devices fell below the hypoglycemic threshold one hour after the insulin challenge and continued to drop even lower towards the end of the experiment for this group. The comparison between the plasma glucagon level of rats treated with the Z-GCN-patch and sham patch was also demonstrated in FIG. 19D. A remarkable spike of plasma glucagon was observed in the Z-GCN-patch group when the blood glucose was in the hypoglycemic range. In contrast, rats treated with the sham patch, showed no significant change in plasma glucagon levels during the experiment. These results confirmed the glucose-responsive delivery of Z-GCN from the MN patch and its ability to effectively prevent hypoglycemia for up to 2 hours in T1D rats against an overdose insulin challenge.

There is provided a matrix microneedle patch device for monitoring and treating high blood sugar or preventing low blood sugar. In an embodiment the microneedles have a length of about 300 μm to about 1000 μm, for example about 800 μm. In another embodiment the microneedles have a base of about 50 μm to about 300 μm, for example about 100 μm. In a further embodiment the microneedles have a needle pitch of about 500 μm to about 2000 μm, for example about 1000 μm. The natural polymeric backbones may be any one or combination of hyaluronan, cellulose, chitosan, chitin, alginate, collagen, gelatin, xanthan, or a combination thereof.

The synthetic polymeric backbones may be any one or combination of polyolefins, polyvinyls, polyesters, polyanhydrides, polyacrylates, polyurethanes, polyamides, poly(3,4-ethylenedioxythiophene) polystyrene sulfonate, polyacetylene, polypyrrole, polyindole and polyaniline.

The glucose-sensing molecules containing glucose-responsive moieties may be any one or combination of lectin, synthetic phenylboronic acid (PBA) and boronic acid containing compounds bearing polymerizable groups.

The glucose-reducing agent may be peptide hormones that reduce blood glucose levels. These peptide hormones that reduce blood glucose levels include anyone or combination of insulin, GLP-1, GLP-1 analogues, pramlintide, gama-aminobutyric acid, glucose-dependent insulinotropic polypeptide and GLP.

The glucose-responsive moieties may be selected to exhibit higher interaction with glucose in response to high glucose level causing swelling of the microneedle and rapid release of the blood glucose-reducing agent.

The blood glucose-raising agents may be peptide hormones that increases blood glucose levels. These peptide hormones that increases the blood glucose levels include anyone or combination of glucagon, glucagon analogues, epinephrine, and epinephrine analogues, norepinephrine and norepinephrine analogues.

The glucose-responsive moieties may be selected to exhibit lower interaction with glucose in response to low blood glucose levels causing the interaction of the glucose-responsive moieties with catechol components and rapid release of the blood glucose-raising agent.

In embodiments the present disclosure provides a kit, comprising at least two matrix microneedle patches one of the patches containing the glucose-reducing agents and the other containing the blood glucose-raising agents, and instructions for a user to affix the patches to their body.

In embodiments a matrix microneedle patch device for treating high blood sugar or preventing low blood sugar is provided, comprising:
a base;
an array of microneedles extending from, and away, from said base, each microneedle of said array and said base being made of a crosslinked mixture of natural and/or synthetic polymeric backbones, catechol containing molecules conjugated to a first preselected fraction of the natural and/or synthetic polymeric backbones and glucose-sensing molecules containing glucose-responsive moieties conjugated to a second preselected fraction of the natural and/or synthetic polymeric backbones with the first and second fractions being crosslinked via catechol-catechol crosslinkages and catechol-glucose-sensing molecules crosslinkages in an absence of chemical crosslinking agents; and
blood glucose-reducing agents entrapped within the crosslinked polymeric backbones or blood glucose-raising agents non covalently bonded to catechol moieties in the catechol containing molecules.

In embodiments there is provided a matrix microneedle patch device for monitoring and treating high blood sugar or preventing low blood sugar, comprising:
a base; and
an array of microneedles extending from, and away, from said base, each microneedle of said array and said base being made of a crosslinked mixture of a first fraction of natural and/or synthetic polymeric backbones having catechol containing molecules conjugated thereto and a second fraction of natural and/or synthetic polymeric backbones and glucose-sensing molecules containing glucose-responsive moieties conjugated thereto, the first and second fractions being crosslinked via catechol-catechol crosslinkages and catechol-glucose-sensing molecules crosslinkages in an absence of chemical crosslinking agents; and
blood glucose-reducing agents entrapped within the crosslinked polymeric backbones or blood glucose-raising agents non covalently bonded to catechol moieties in the catechol containing molecules.

In embodiments there is provided a matrix microneedle patch device having contained therein a composition for treating high blood sugar or preventing low blood sugar, compromising:
a microneedle patch with a base;
an array of microneedles extending from and away from said base;
blood glucose-raising agents or blood glucose-reducing agents loaded into the patch;
each microneedle of said array and said base being made of a mixture of natural and/or synthetic polymeric backbones;
catechol containing molecules covalently conjugated to the natural and/or synthetic polymeric backbones, wherein, the catechol is either covalently or non-covalently coupled to one another that form crosslinkages between the polymeric backbones;
glucose-sensing molecules containing glucose-responsive moieties covalently conjugated to the natural and/or synthetic polymeric backbones that interact with catechol containing molecules, wherein the glucose-responsive moieties reversibly interact or dissociate with the catechol containing molecules response of the concentration in the of a diol-containing compound, and
wherein when blood glucose-reducing agents are present they are entrapped and prevents blood glucose-raising beyond a preselected blood glucose level, and when the blood glucose-reducing agents are present they are non-covalently bonded to catechol moieties in the catechol containing molecules.

In embodiments the glucose-sensing molecules may include, but are not limited to, lectin, synthetic phenylboronic acid (PBA) and boronic acid containing compounds bearing polymerizable groups. For example, non-limiting examples of lectins may include, but are not limited to, concanavalin or boronic acid containing compounds. The glucose-responsive molecules may be synthetic phenylboronic acid (PBA) or boronic acid containing compounds bearing various polymerizable groups including (meth)acrylates, (meth)acrylamides and styrene. Examples of such glucose responsive monomers (e.g., PBA containing (meth)acrylamides) include, but are not limited to, 4-mercaptophenylboronic acid, phenylboronic acid, 3-alkylamidophenylboronic acid, 4 carboxyphenylboronic acid, 4-acetamido-3-fluorophenylboronic acid, 2-hydroxymethylphenylboronic acid (benzoboroxole), 4-nitrophenylboronic acid, 3-acetamido-6-heptafluoropropylphenylboronic acid, 4-vinylphenylboronic acid, 3-acrylamidophenylboronic acid, 4-(1,6-dioxo-2,5-diaza-7-oxamyl)phenylboronic acid, 2-dimethylaminomethyl-5-vinylphenylboronic acid, 4-(N allylsulfamoyl)phenylboronic acid, 4-(3-butenylsulfonyl)phenylboronic acid, 3-(acrylamido)-phenylboronic acid (ABA) and 4-Acrylamido-3-fluorophenylboronic acid (AFBA).

In an embodiment there is provided a method of producing a matrix microneedle patch containing blood glucose-reducing agents or blood glucose-raising agents, comprising:
providing a mold of preselected size for receiving polymer solutions therein, which once the patch is produced it has a base section and microneedles extending away from said base section;
mixing a first polymer solution of natural and/or synthetic polymeric backbones having catechol containing molecules covalently conjugated to the polymeric backbones with blood glucose-reducing agents or blood glucose-raising agents and a second polymer solution of natural and/or synthetic polymeric backbones containing glucose-responsive moieties covalently conjugated to the polymeric backbones that interact with the catechol containing molecules such that the natural and/or synthetic polymeric backbones crosslink by auto-oxidation of catechol groups and reversible interactions between the glucose-sensing molecule and the catechol functional groups in the absence of any chemical crosslinking agent; and
wherein when blood glucose-reducing agents are present they are entrapped and prevent the blood glucose level rising beyond a preselected blood glucose level, and when the blood glucose-increasing agents are present they are non-covalently bonded to catechol moieties in the catechol containing molecules and increase blood glucose levels and prevents reduction of blood glucose beyond a preselected blood glucose level. The natural and/or synthetic polymeric backbones are crosslinked upon mixing of the polymer solutions at a specific weight ratio, wherein a weight ratio of the catechol-conjugated natural and/or synthetic polymers to glucose-sensing molecules conjugated natural and/or synthetic polymeric backbones are about 0.25:3 to about 3:0.25. Alternatively, the weight ratio is about 1.1 to about 3:1. Alternatively, weight ratio is about 2:1.

1. (canceled)

2. A matrix microneedle patch device for monitoring and/or treating high blood sugar or preventing low blood sugar, comprising:
a base;
an array of microneedles extending from, and away, from said base, each microneedle of said array and said base being made of a crosslinked mixture of a first fraction of natural and/or synthetic polymeric backbones having catechol containing molecules conjugated thereto and a second fraction of natural and/or synthetic polymeric backbones and glucose-sensing molecules containing glucose-responsive moieties conjugated thereto, the first and second fractions being crosslinked via catechol-catechol crosslinkages and catechol-glucose-sensing molecules crosslinkages in an absence of chemical crosslinking agents; and
blood glucose-reducing agents entrapped within the crosslinked polymeric backbones or blood glucose-raising agents non covalently bonded to catechol moieties in the catechol containing molecules.

3. A matrix microneedle patch device having contained therein a composition for treating high blood sugar or preventing low blood sugar, comprising:
a microneedle patch with a base;
an array of microneedles extending from and away from said base;
blood glucose-raising agents or blood glucose-reducing agents loaded into the patch;
each microneedle of said array and said base being made of a mixture of natural and/or synthetic polymeric backbones;
catechol containing molecules covalently conjugated to the natural and/or synthetic polymeric backbones, wherein, the catechol is either covalently or non-covalently coupled to one another that form crosslinkages between the polymeric backbones;
glucose-sensing molecules containing glucose-responsive moieties covalently conjugated to the natural and/or synthetic polymeric backbones that interact with catechol containing molecules, wherein the glucose-responsive moieties reversibly interact or dissociate with the catechol containing molecules in response of the concentration of a diol-containing compound; and
wherein when blood glucose-reducing agents are present they are entrapped and prevents blood glucose-raising beyond a preselected blood glucose level, and when the blood glucose-reducing agents are present they are non-covalently bonded to catechol moieties in the catechol containing molecules.

4. The matrix microneedle patch device according to claim 2, wherein the natural polymeric backbones are selected from the group consisting of hyaluronan, cellulose, chitosan, chitin, alginate, collagen, gelatin, xanthan, and a combination thereof.

5. The matrix microneedle patch device according to claim 2, wherein the synthetic polymeric backbones are selected from the group consisting of polyolefins, polyvinyls, polyesters, polyanhydrides, polyacrylates, polyurethanes, polyamides, poly(3,4-ethylenedioxythiophene) polystyrene sulfonate, polyacetylene, polypyrrole, polyindole, polyaniline or combinations or copolymers thereof.

6. The matrix microneedle patch device according to claim 2, wherein the glucose-sensing molecules are any one or combination of lectin, synthetic phenylboronic acid (PBA) and boronic acid containing compounds bearing polymerizable groups.

7. The matrix microneedle patch device according to claim 2, wherein the glucose-reducing agent is selected from the group consisting of peptide hormones that reduce blood glucose levels.

8. The device according to claim 7, wherein the peptide hormones that reduce blood glucose levels include any one or combination of insulin, GLP-1, GLP-1 analogues, pramlintide, gama-aminobutyric acid, glucose-dependent insulinotropic polypeptide and GLP.

9. The matrix microneedle patch device according to claim 2, wherein the glucose-responsive moieties exhibit higher interaction with glucose in response to high glucose level causing swelling of the microneedle and rapid release of the blood glucose-reducing agent.

10. The matrix microneedle patch device according to claim 2, wherein the glucose-raising agents are selected from the group consisting of peptide hormones that increases blood glucose levels.

11. The device according to claim 10, wherein the peptide hormones that increases the blood glucose levels include any one or combination of glucagon, glucagon analogues, epinephrine, and epinephrine analogues, norepinephrine and norepinephrine analogues.

12. The matrix microneedle patch device according to claim 2, wherein the glucose-responsive moieties exhibit lower interaction with glucose in response to low blood glucose levels causing the interaction of the glucose-responsive moieties with catechol components and rapid release of the blood glucose-raising agent.

13. A kit, comprising:
at least two matrix microneedle patch devices according to claim 2 with one of said patch devices containing the glucose-reducing agents and the other containing the blood glucose-raising agents, and instructions for a user to affix the patch devices to their body.

14. A method of producing a matrix microneedle patch containing blood glucose-reducing agents or blood glucose-raising agents, comprising:
providing a mold of preselected size for receiving polymer solutions therein, which once the patch is produced it has a base section and microneedles extending away from said base section;
mixing a first polymer solution of natural and/or synthetic polymeric backbones having catechol containing molecules covalently conjugated to the polymeric backbones with blood glucose-reducing agents or blood glucose-raising agents and a second polymer solution of natural and/or synthetic polymeric backbones containing glucose-responsive moieties covalently conjugated to the polymeric backbones that interact with the catechol containing molecules such that the natural and/or synthetic polymeric backbones crosslink by auto-oxidation of catechol groups and reversible interactions between the glucose-sensing molecule and the catechol functional groups in the absence of any chemical crosslinking agent; and
wherein when blood glucose-reducing agents are present they are entrapped and prevent the blood glucose level rising beyond a preselected blood glucose level, and when the blood glucose-increasing agents are present they are non-covalently bonded to catechol moieties in the catechol containing molecules and increase blood glucose levels and prevents reduction of blood glucose beyond a preselected blood glucose level.

15. The method according to claim 14, wherein the natural and/or synthetic polymeric backbones are crosslinked upon mixing of the polymer solutions at a specific weight ratio, wherein a weight ratio of the catechol-conjugated natural and/or synthetic polymers to glucose-sensing molecules conjugated natural and/or synthetic polymeric backbones are about 0.25:3 to about 3:0.25.

16. The method according to claim 15, wherein the weight ratio is about 1.1 to about 3:1.

17. The method according to claim 16, wherein the weight ratio is about 2:1.

18. The matrix microneedle patch device according to claim 3, wherein the natural polymeric backbones are selected from the group consisting of hyaluronan, cellulose, chitosan, chitin, alginate, collagen, gelatin, xanthan, and a combination thereof and the synthetic polymeric backbones are selected from the group consisting of polyolefins, polyvinyls, polyesters, polyanhydrides, polyacrylates, polyurethanes, polyamides, poly(3,4-ethylenedioxythiophene) polystyrene sulfonate, polyacetylene, polypyrrole, polyindole, polyaniline or combinations or copolymers thereof.

19. The matrix microneedle patch device according to claim 3, wherein the glucose-sensing molecules are any one or combination of lectin, synthetic phenylboronic acid (PBA) and boronic acid containing compounds bearing polymerizable groups and the glucose-reducing agent is selected from the group consisting of peptide hormones that reduce blood glucose levels.

20. The matrix microneedle patch device according to claim 3, wherein the glucose-sensing molecules are any one or combination of lectin, synthetic phenylboronic acid (PBA) and boronic acid containing compounds bearing polymerizable groups, the glucose-reducing agent is selected from the group consisting of peptide hormones that reduce blood glucose levels and the glucose-raising agents are selected from the group consisting of peptide hormones that increases blood glucose levels.

21. The matrix microneedle patch device according to claim 3, wherein the glucose-responsive moieties exhibit higher interaction with glucose in response to high glucose level causing swelling of the microneedle and rapid release of the blood glucose-reducing agent or the glucose-responsive moieties exhibit lower interaction with glucose in response to low blood glucose levels causing the interaction of the glucose-responsive moieties with catechol components and rapid release of the blood glucose-raising agent.
